SEQUENCE LISTING (5 sequences)

SEQ ID NO: 1 - protein, 8 aa, synthetic construct
GHPGGPQG

SEQ ID NO: 2 - protein, 13 aa, synthetic construct
GGGMGPSGPWGGK

SEQ ID NO: 3 - protein, 6 aa, synthetic construct
PLGLAG

SEQ ID NO: 4 - protein, 16 aa, synthetic construct
GCRDGPQGIWGQDRCG

SEQ ID NO: 5 - protein, 16 aa, synthetic construct
GCRDGPQGIAGQDRCG

(19) United States
(12) Patent Application Publication
(10) Pub. No.: US 2026/0284201 A1
(43) Pub. Date: Sep. 24, 2026

(54) COMPOSITIONS AND METHODS FOR TREATING TENDON AND BONE INJURIES

(71) Applicants: The United States Government as represented by the Department of Veterans Affairs, Washington, DC (US); University of Southern California, Los Angeles, CA (US); The Regents of the University of California, Oakland, CA (US)

(72) Inventors: Charles Edward McKenna, Los Angeles, CA (US); Thomas Kremen, Los Angeles, CA (US)

(21) Appl. No.: 19/479,573

(22) PCT Filed: May 3, 2024
(86) PCT No.: PCT/US2024/027725

§ 371 Date: Oct. 28, 2025

Related U.S. Application Data
(60) Provisional application No. 63/500,463, filed on May 5, 2023.

(51) Int. Cl. A61K 47/54; A61K 47/65; A61P 19/08; C07K 14/495; C07K 14/51; C07K 14/635; C07K 14/705; C07K 14/78

(52) U.S. Cl. CPC A61K 47/548; A61K 47/65; A61P 19/08; C07K 14/495; C07K 14/51; C07K 14/635; C07K 14/705; C07K 14/78; C07K 2319/30

(57) ABSTRACT

In one aspect, the invention relates to synthetic molecules, compositions and pharmaceutical compositions, which are useful for delivering a bioactive moiety to a site of injury. Also disclosed are methods of treating a tendon injury, methods of treating a fracture, methods of treating a bone stress injury, methods of enhancing incorporation of allograft tissues, methods of promoting repair or regeneration of enthesis tissue during or after an orthopedic surgical procedure, and method of treating osteoporosis, osteoarthritis and/or cartilage defects using the disclosed molecules, compositions and pharmaceutical compositions.

### CROSS-REFERENCE TO RELATED APPLICATIONS

This application claims the benefit of U.S. Provisional Patent Application No. 63/500,463, filed May 5, 2023, which is incorporated by reference herein in its entirety.

### STATEMENT REGARDING FEDERALLY SPONSORED RESEARCH

This invention was made with government support under IK2BX005199 awarded by the Department of Veterans Affairs. The government has certain rights in the invention.

### SEQUENCE LISTING STATEMENT

The Sequence Listing submitted Mar. 3, 2024 as a text file named “37759_0536P1_SL.xml,” created on Mar. 3, 2024, and having a size of 5,433 bytes is hereby incorporated by reference pursuant to 37 C.F.R. § 1.52(e)(5).

### BACKGROUND

There are 32 million musculoskeletal injuries in the United States annually of which 45% involve tendons or ligaments such as rotator cuff tendon tears and anterior cruciate ligament (ACL) tears¹. These two common injuries that are often treated surgically and constitute a significant healthcare burden in the United States. Approximately 250,000 RCT repairs are performed each year in the US alone and it has been estimated that successful rotator cuff repairs in the US result in a lifetime societal savings of $3.44 billion^{2;3}. Unfortunately, on average 12% to 40% of rotator cuff repairs will fail to heal, and failure of up to 94% of repairs has been reported when rotator cuff tears are larger than 2 cm in size or affect patients greater than 60 years of age³⁻⁵, which is a noteworthy challenge since both of these factors are commonly encountered in clinical practice. A critical feature related to this high rate of observed tendon repair failure is that, currently, tendon-to-bone repairs heal via the formation of disorganized fibrotic tissue that fails to restore the native tissue architecture, and this abnormal reparative tissue is compositionally and mechanically inferior to the native tendon and enthesis tissue^{1;6-8}

In general, the biology of tissue regeneration involves cells, extracellular matrix (ECM) and growth factors. Cells are in relative abundance given the close proximity of decorticated bone, periosteum, bursal adipose and often synovial tissues at the time of a tendon-bone repair and the available pool of local mesenchymal progenitor cells are certainly capable of proliferating and producing ECM when appropriately exposed to various trophic factors. While certain growth factors such as bone morphogenetic proteins (BMPs) or transforming growth factor-beta (TGFβ)⁸⁻¹⁵ have been reported to improve both the biomechanical strength and tissue organization of the repaired enthesis, their clinical applicability continues to be severely limited due to a lack of effective delivery strategies.

Delivering growth factors to the site of tendon repair is an attractive strategy to incite the ingress of local progenitor cells and trigger tissue regeneration. Current clinical approaches to growth factor delivery are limited to supraphysiologic doses administered at the gross tissue level at one point in time (i.e. intra-operative delivery). Unfortunately, there is no current methodology to i) effectively deliver physiologically-relevant levels of growth factors at the cellular level, ii) target delivery to the site of surgical repair, and iii) deliver growth factors over multiple time points.

What is needed are molecules, compositions and pharmaceutical compositions and methods for targeting a molecule to a site of injury or site of surgical repair. Also needed are methods of treating a tendon injury, methods of treating a fracture, methods of treating an osteotomy and methods of treating a bone stress injury.

### BRIEF SUMMARY

Described herein are molecules, compositions and pharmaceutical compositions as well as methods for targeting a molecule to a site of injury. Further described herein are molecules, compositions and pharmaceutical compositions for treating a tendon injury, a fracture, and/or a bone stress injury. Also described herein are molecules, compositions and pharmaceutical compositions for enhancing incorporation of allograft tissues and for promoting repair or regeneration of enthesis during or after an orthopedic surgical procedure.

Disclosed herein are molecules comprising i) a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are compositions comprising a molecule, wherein the molecule comprises a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are pharmaceutical compositions comprising a molecule, wherein the molecule comprises i) a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are methods of targeting a molecule to a site of injury comprising administering a molecule, wherein the molecule comprises i) a bioactive moiety; ii) a cleavable linker; and iii) a bisphosphonate moiety (BP): wherein the BP moiety has a P-C-P structure, and wherein the cleavable linker links the bioactive moiety to the BP moiety.

Disclosed herein are methods of treating a tendon injury, a fracture, and/or a bone stress injury and/or promoting soft tissue to bone healing comprising administering a molecule, wherein the molecule comprises i) a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are methods of enhancing incorporation of allograft tissues during or after an orthopedic surgical procedure comprising administering a molecule, wherein the molecule comprises i) a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are methods of promoting repair or regeneration of enthesis during or after an orthopedic surgical procedure comprising administering a molecule, wherein the molecule comprises i) a bioactive moiety; ii) a cleavable linker; and iii) a bisphosphonate moiety (BP); wherein the BP moiety has a P-C-P structure, and wherein the cleavable linker links the bioactive moiety to the BP moiety.

Disclosed herein are methods of treating osteoporosis, osteoarthritis and/or cartilage defects comprising administering a molecule, wherein the molecule comprises i) a bioactive moiety: ii) a cleavable linker; and iii) a bisphosphonate moiety (BP); wherein the BP moiety has a P-C-P structure, and wherein the cleavable linker links the bioactive moiety to the BP moiety.

### BRIEF DESCRIPTION OF THE DRAWINGS

The accompanying drawings, which are incorporated in and constitute a part of this specification, illustrate several aspects of the disclosed method and compositions and together with the description, serve to explain the principles of the disclosed method and compositions.

FIGS. 1A-1B is a diagram depicting BP-F-Q (OSF-3) molecule mechanism of action. FIG. 1A shows that administered BP-F-Q localizes to exposed bone surface at the tendon (T) repair site. In the absence of Ctsk, fluorescence from the fluorophore (F) is suppressed by the covalently linked quenching dye (Q). FIG. 1B shows that in the presence of local activated osteoclasts, Ctsk at the site of tendon repair specifically cleaves the linker peptide (dashed blue line) allowing detection of an external fluorescence signal from F.

FIGS. 2A-2D are pictures showing surgical methods. FIG. 2A shows transosseous and transtendinous stitch placement. FIG. 2B shows sharp transection of Achilles tendon at bone attachment. FIG. 2C shows posterior-superior calcaneus burr for decortication. FIG. 2D shows repair of transected tendon to decorticated calcaneus.

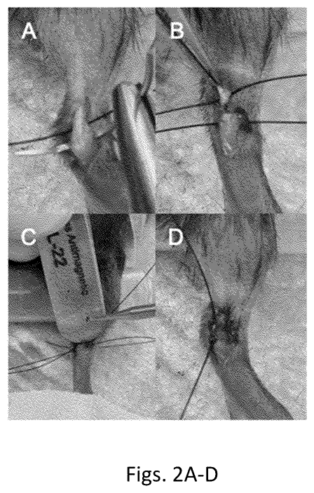

FIGS. 3A-3B are graphs showing the mean fluorescence on-target signal (FIG. 3A) and off-target signal (FIG. 3B) over time among systemic, local and sham treatment groups (n=7 per group) who received AF647-ZOL. Error bars represent standard deviation from the mean value. Both ** and * denote a significant difference between Sham and Local groups (p<0.01, p<0.05) respectively. Both ## and # denote a significant difference between Sham and Systemic groups (p<0.01, p<0.05) respectively.

FIGS. 4A-4C show representative image of fluorescence signal on POD 10 among animals treated with AF647-ZOL.

FIGS. 5A and 5B are graphs showing the mean fluorescence on-target signal (FIG. 5A) and off-target signal (FIG. 5B) over time among repair (systemic) and sham treatment groups (n=3 per group) who received Cat K 680 FAST. Error bars represent standard deviation from the mean value. ** and * denote a significant difference between Sham and Repair groups (p<0.01, p<0.05) respectively.

FIGS. 6A-6B are representative image of fluorescence signal on POD 4 among sham (FIG. 6B) and Achilles tendon repair (FIG. 6A) animals treated with Cat K 680 FAST.

FIGS. 7A-7C shows representative in vivo fluorescent imaging at post-operative day 10 for local (FIG. 7A), systemic (FIG. 7B), and sham (FIG. 7C) treatment groups. Note the hindfoot on-target signal in the repaired local and systemic treated animal and the forefoot off-target signal in the sham animal.

FIGS. 8A-8B are graphs showing mean fluorescence on-target signal (FIG. 8A) and off-target signal (FIG. 8B) over time among systemic, local and sham treatment groups (n=7 per group) who received OFS-3. Error bars represent standard deviation from the mean value. Both ** and * denote a significant difference between Sham and Local groups (p<0.01, p<0.05) respectively. Both ## and # denote a significant difference between Sham and Systemic groups (p<0.01, p<0.05) respectively.

FIGS. 9A-9D show hindfoot sections at 4× magnification stained with H&E and SOFG (FIG. 9A) and immunohistochemistry for Ctsk (FIG. 9B) in normal uninjured mice and stained with H&E and SOFG (FIG. 9C) and immunohistochemistry-stained for Ctsk (FIG. 9D) in POD 4 after Achilles tendon-to-bone repair. # denotes Distal Achilles tendon just proximal to calcaneal enthesis (FIGS. 9A, B). } denotes normal cortical and cancellous bone of posterior-superior region of the calcaneus (FIGS. 9A, B). Arrow denotes the transected residual proximal stump of repaired Achilles tendon with Ctsk positive IHC staining (FIGS. 9C, D). * denotes the suture hole created at posterior region of the calcaneus during repair. [ ] denotes the decorticated region of posterior-superior calcaneus with significant Ctsk positive IHC staining (FIGS. 9C, D).

FIGS. 10A-10B show mouse hindfoot sections at 4× magnification at POD 7 (FIG. 10A) and POD 28 (FIG. 10B) immunohistochemistry stained for Ctsk. Note the absence of the residual Achilles tendon (RAT) and the absence of osteoclastic activity at POD 28 (FIG. 10B). S=suture fragments contained within this tissue section at POD 7 (FIG. 10A). Scale bar: 0.5 mm. The arrow denotes osteoclast resorption of bone at the site of tendon-bone repair. The area between the [ ] denotes the reparative tissue at the tendon-bone junction (FIGS. 10A and 10B). The { identifies an area of osteoclastic resorption at the posterior calcaneus which may be occurring due to altered hindfoot weight bearing in the first week of recovery following the Achilles tendon repair procedure.

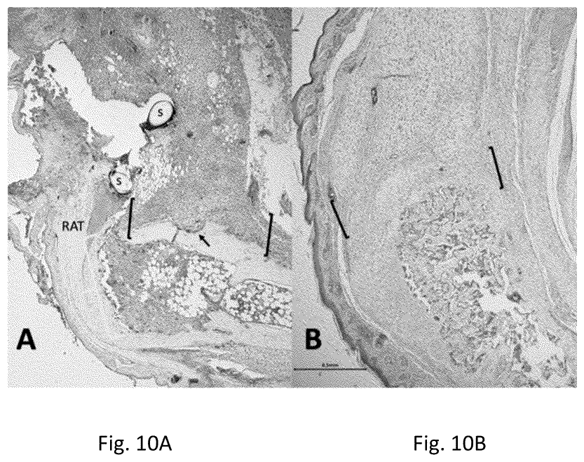

FIG. 11 is a bar graph showing activation of BMP receptors (by measuring bone response element (BRE) expression) in cells treated with (1) Phosphate Buffered Saline (control), (2) rhBMP-2, (3) variant BMP-2, (4) Ctsk-digested BP-vBMP-2 or (5) undigested BP-vBMP-2. Luciferase expression from BRE plasmid was detected and normalized to control Rinella plasmid luciferase expression.

### DETAILED DESCRIPTION

The disclosed method and compositions may be understood more readily by reference to the following detailed description of particular aspects and the Examples included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular aspects only and is not intended to be limiting.

### A. Definitions

The terminology used herein is for the purpose of describing particular aspects only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

The word “or” as used herein means any one member of a particular list and also includes any combination of members of that list.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine: D aspartic acid; E, glutamic acid; F, phenylalanine; G, glycine: H histidine; I isoleucine; K, lysine; L, leucine; M, methionine; N, asparagine; P, proline; Q, glutamine; R, arginine; S, serine: T, threonine: V, valine; W, tryptophan; and Y, tyrosine. All alpha amino acids except glycine can exist in either of two enantiomers, called L-amino acids or D-amino acids, which are mirror images of each other.

As used herein, the term “amino acid” refers to an organic molecule with a basic amino group (—NH2), an acidic carboxyl group (—COOH), a proton and a variable ‘R’ group bound to an sp3 hybridized central carbon atom. The N-terminus of a peptide has a free amino group (—NH2). The C-terminus of a peptide has a free carboxyl group (—COOH).

As used herein, the terms “D-amino acid” or “amino acid in the D-form” refer to amino acids where the stereogenic carbon alpha to the amino group has the D-configuration. D amino acid is the enantiomer of an amino acid that is capable of rotating plane polarized light clockwise (right-hand side). D-amino acids are designated with the prime (′) symbol.

As used herein, the terms “L-amino acid” or “amino acid in the L-form” refer to amino acids where the stereogenic carbon alpha to the amino group has the L-configuration. L amino acid is the enantiomer of an amino acid that is capable of rotating plane polarized light anticlockwise (left-hand side). With the exception of achiral glycine, natural amino acids have the L configuration.

“Peptide” as used herein refers to any peptide, oligopeptide, polypeptide, gene product, expression product, or protein. A peptide is comprised of consecutive amino acids. The term “peptide” encompasses naturally occurring or synthetic molecules. A residue of a peptide is an amino acid. As used herein if an amino acid is said to be in the 5^{th} position, it is the 5^{th} amino acid from the N-terminus of the peptide.

By “fragment” is meant a portion of a polypeptide or nucleic acid molecule, such as, but not limited to, a truncation mutant. This portion contains, preferably, at least about 5%, 10%, 20%, 30%, 40%, 50%, 60%, 70%, 80%, 90%, 95% of the entire length of the reference nucleic acid molecule or polypeptide. For example, a fragment of a polypeptide may contain about 5, about 10, about 15, about 20, about 25 or about 30 or more amino acids. In another example, a fragment of a polypeptide may contain about 1000, about 1500, about 2000, about 2500 or about 3000 or more amino acids.

As used herein, the term “epitope” refers to a localized region on the surface of an antigen capable of eliciting an immune response.

As used herein, “sample” is meant to mean an animal; a tissue or organ from an animal: a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties are those in which: (a) the hydrophilic residue, e.g. seryl or threonyl, is substituted for (or by) a hydrophobic residue, e.g., leucyl, isoleucyl, phenylalanyl, valyl or alanyl; Tryptophan, Tyrosinyl (b) a cysteine or proline is substituted for (or by) any other residue; (c) a residue having an electropositive side chain, e.g., lysyl, arginyl, or hystidyl, is substituted for (or by) an electronegative residue, e.g. glutamyl or aspartyl; or (d) a residue having a bulky side chain, e.g., phenylalanine, is substituted for (or by) one not having a side chain, e.g., glycine, in this case, or (e) by increasing the number of sites for sulfation and/or glycosylation.

TABLE 1
Amino Acid Substitutions

| | Non-limiting Exemplary |
|---|---|
| Original Residue | Conservative Substitutions |
| Ala | Ser |
| Arg | Gly; Gln; Lys |
| Asn | Gln; His |
| Asp | Glu |
| Cys | Ser |
| Gln | Asn; Lys |
| Glu | Asp |
| Gly | Ala |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

TABLE 2
Amino Acid Abbreviations

| Amino Acid | Abbreviations |
|---|---|
| Alanine | Ala (A) |
| Allosoleucine | AIle |
| Arginine | Arg (R) |
| Asparagine | Asn (N) |
| Aspartic Acid | Asp (D) |
| Cysteine | Cys (C) |
| Glutamic Acid | Glu (E) |
| Glutamine | Gln (Q) |
| Glycine | Gly (G) |
| Histidine | His (H) |
| Isolelucine | Ile (I) |
| Leucine | Leu (L) |
| Lysine | Lys (K) |
| Phenylalanine | Phe (F) |
| Praline | Pro (P) |
| Pyroglutamic Acid | PGlu (U) |
| Serine | Ser (S) |
| Threonine | Thr (T) |
| Tyrosine | Tyr (Y) |
| Tryptophan | Trp (W) |
| Valine | Val (V) |

It is understood that one way to define the variants and derivatives of the disclosed proteins herein is to define them in terms of homology/identity to specific known sequences. Specifically disclosed are variants of peptides herein disclosed which have at least, 70% or at least 75% or at least 80% or at least 85% or at least 90% or at least 95% or at least 96% or at least 97% or at least 98% or at least 99% identity to the peptides specifically recited herein. Those of skill in the art readily understand how to determine the identity of two proteins.

The polypeptides can be modified by either natural processes, such as posttranslational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, “osteochondral factor” refers to biological compounds that promote bone and/or cartilage repair and/or regeneration. Osteochondral factors include, but are not limited to, NELL-1, TGF-β1, TGF-β2, TGF-β, BMP-1, BMP-2, BMP-3, BMP-4, BMP-5, BMP-6, BMP-7, BMP-8a, BMP-8b, BMP-10, BMP-11, BMP-15, or rhBMP.

As used herein. “osteogenic factor” refers to biological compounds that promote new bone and/or new cartilage formation. Buser Z et al. Eur Spine J. 2017 November: 26(11):2763-2772. Osteogenic factors include, but are not limited to, oxy133 and oxysterol-133.

As used herein, “tenogenic factor” refers to biological compounds that promote tendon repair and/or regeneration. Tenogenic factors include, but are not limited to TGFβ-1, TGFβ-2, TGF-β, FGF, CTGF, BMP-12, BMP-13, BMP-14, CCN1 or WISP-1.

As used herein, “ECM protein” refers to proteins that are present in the extracellular matrix. The extracellular matrix is a three-dimensional network of extracellular macromolecules and minerals (including hydroxyapatite) that provide structural and biochemical support to surrounding cells. ECM proteins include, but are not limited to collagen, elastin, fibrin, fibronectin, gelatin, laminin, integrin. CCN1, WISP-1 or CTGF.

As used herein, “tendon injury” refers to damage or inflammation of the connective tissue that links muscle to bone. It can be caused by overuse, degeneration, or trauma. As used herein, treating a tendon injury includes promoting soft tissue to bone healing.

As used herein, “fracture” refers to a break in a bone.

As used herein, “bone stress injury” refers to overuse injuries that are typified by a gradual onset of pain with activity. Examples of bone stress injuries include, but are not limited to, stress reactions and stress fractures. Bone stress injuries develop in response to repetitive loads applied to bone. Bone stress injuries are often seen on MRI as high signal intensity.

As used herein, treating “osteointegration of an implantable prosthesis” includes enhanced healing of bone to an implant material (metal, ceramic, etc.) when that implant is implanted into bone or dental applications.

As used herein, treating “osteointegration of allograft tissue” includes, but is not limited to, osteochondral allografts and bone allografts.

As used herein, “osteoarthritis” includes, but is not limited to, degenerative cartilage damage within a joint or intervertebral disc space, including damage from abnormal bone remodeling. Treatment of osteoarthritis includes, but is not limited to, treatment of osteoarthritic bone abnormalities, including subchondral bone and bone marrow edema as well as osteoarthritic cartilage abnormalities as the bone and cartilage abnormalities associated with osteoarthritis are related.

As used herein “cartilage defects” includes, but is not limited to, areas of damaged cartilage surrounded by relatively more healthy cartilage. In some aspects, the cartilage defect can be a defect or injury to articular or hyaline cartilage, fibrocartilage oe elastic cartilage. The cartilage defects can be the result of traumatic mechanical destruction or progressive mechanical destructions. In some aspects, the cartilage defect can be in a specific, localized area of damage to the cartilage.

“Dose” or “dosage” as used herein refers to a specific quantity of a therapeutic agent, such as a molecule, composition or pharmaceutical composition that is taken at specific times.

As used herein, “treat” is meant to mean administer one of the disclosed compositions to a subject, such as a human or other mammal (for example, an animal model), that has atherosclerosis, in order to prevent or delay a worsening of the effects of the disease or condition, or to partially or fully reverse the effects of the disease.

As used herein, “effective amount” is meant to mean a sufficient amount of one or more of the molecules, compositions or pharmaceutical compositions disclosed herein to provide the desired effect. For example, an effective amount of one or more of the molecules, compositions or pharmaceutical compositions disclosed herein can be an amount that provides a therapeutic affect and provides sustained therapeutic effects after withdrawal of the treatment. An effective amount of one or more of the molecules, compositions or pharmaceutical compositions disclosed herein can be an amount that is able to cause a benefit illustrated by, for example, promoting healing of tendon injuries, speeding up recovery time, improving biomechanical properties (such as strength or toughness) of the bone, cartilage and/or tendon, and/or improving tissue organization of bone, cartilage and/or tendon, as well as an amount that allows for a sustained therapeutic effect after withdrawal of the molecule, composition or pharmaceutical composition. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The terms “vector” or “construct” refer to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids.

Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. COMPOSITIONS

### 1. Molecules

Non-limiting examples of the molecules of the disclosure are provided herein.

Disclosed herein are molecules comprising: i) a bioactive moiety, ii) a cleavable linker, and iii) a bisphosphonate moiety, wherein the BP moiety has a P-C-P structure and wherein the cleavable linker links the bioactive moiety to the BP moiety.

i. Bioactive Moiety

As used herein, “bioactive moiety” is meant a part of a molecule that has or can elicit an action in the body of a subject or a physiological or pharmacological response in the body of a subject.

In some aspects, the bioactive moiety is an osteochondral factor, an osteogenic factor, a tenogenic factor or an Extracellular Matrix (ECM) protein. In some aspects, the ECM protein is a collagen, elastin, fibrin, fibronectin, gelatin, laminin, integrin, CCN1, WISP-1 or CTGF.

In some aspects, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects, the osteochondral factor is NELL-1 or BMP-2.

In some aspects, the bioactive moiety is an osteogenic factor and the osteogenic factor is oxy133 or oxysterol-133.

In some aspects, the bioactive moiety is a tenogenic factor and the tenogenic factor is TGFβ-1, TGFβ-2, TGF-β, FGF, CTGF, BMP-12, BMP-13, BMP-14, CCN1 or WISP-1. In some aspects, the tenogenic factor is TGFβ-1 or TGFβ-2.

In some aspects, the bioactive moiety is a hormone. In some aspects, the hormone is parathyroid hormone (e.g., teriparatide, Natpara). In some aspects, the bioactive moiety is parathyroid hormone-related protein.

In some aspects, the bioactive moiety is a growth differentiation factor. In some aspects, the growth differentiation factor is bone morphogenic protein 11 (BMP-11).

In some aspects, the bioactive moiety is activin receptor-like kinase-1-Fc (ALK1-Fc, dalantercept). ALK1-Fc is a chimeric protein with ALK1 receptor domain combined with the Fc portion of human IgG and serves as a ligand trap for ALK1 ligands BMP9 and BMP10.

In some aspects, the bioactive moiety is activin receptor-like kinase-4-Fc (ALK4-Fc). ALK4-Fc is a chimeric protein with ALK4 receptor domain combined with the Fc portion of human IgG and serves as a ligand trap for ALK4 ligands activins, GDF8 and GDF11.

ii. Cleavable Linker

As used herein. “cleavable linker” is meant a peptide linker that is capable of being cleaved. In some aspects, the cleavable linker can be enzymatically cleaved (i.e. enzymatically cleavable linkers). In some aspects, the cleavable linker can be a protease-sensitive peptide linker, acid sensitive hydrazone linker, or a glutathione-sensitive disulfide linker. Examples of cleavable linkers include, but are not limited to peptides which are capable of being cleaved by the enzyme cathepsin K (cathepsin K-sensitive linker) or by the matrix metalloproteinase-2 enzyme (MMP linker). Cleavable linkers can also include peptide linkers that are substrates for other matrixmetalloproteinase enzymes such as MMP-1, MMP-3, MMP-9, MMP-10 and MMP-13. Examples of cleavable linkers can include, but are not limited to the cleavable linkers in Table 3.

TABLE 3
Cleavable Linkers

| | | SEQ |
|---|---|---|
| Enzyme | Amino acid sequence | ID NO: |
| Cathepsin K | GHPGGPQG | 1 |
| Cathepsin K | GGGMGPSGPWGGK | 2 |
| MMP | PLGLAG | 3 |
| MMP | GCRD-GPQGIWGQ-DRCG | 4 |
| MMP | GCRD-GPQGIAGQ-DRCG | 5 |

Also disclosed a variants or derivatives of the cleavable linkers disclosed herein. As used herein, the term “analog” is used interchangeably with “variant” and “derivative.” Variants and derivatives are well understood to those of skill in the art and can involve amino acid sequence modifications. Such amino acid sequence modifications typically fall into one or more of three classes: substantial; insertional; or deletional variants. Insertions include amino and/or carboxyl terminal fusions as well as intrasequence insertions of single or multiple amino acid residues. Insertions ordinarily are smaller insertions than those of amino or carboxyl terminal fusions, for example, on the order of one to four residues. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Substitutions, deletions, insertions or any combination thereof may be combined to arrive at a final derivative or analog. Substitutional variants are those in which at least one residue has been removed and a different residue inserted in its place. Such substitutions generally are made in accordance with Tables 1 and 2 and are referred to as conservative substitutions.

In some aspects, the cleavable linker is a cathepsin-K-sensitive peptide linker. In some aspects, the cathepsin-K-sensitive peptide linker is GHPGGPQG (SEQ ID NO: 1) or GGGMGPSGPWGGK (SEQ ID NO: 2).

In some aspects, the cleavable linker is an MMP cleavable linker. In some aspects, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. Nos. 10,383,912, 10,688,193 both of which are hereby incorporated by reference in their entireties.

iii. Bisphosphonate Moiety

As used herein, “bisphosphonate moiety” refers to a molecule characterized by two C-P bonds. When the two C-P bonds share a single carbon atom (P-C-P), they are deemed to be analogues of pyrophosphate (P-O-P) and are called geminal bisphosphonates (so-called because the carbon is at the central or geminal position). The P-C-P bonds of the geminal bisphosphonate are stable to heat and most chemical reagents and are completely resistant to enzymatic hydrolysis. In some aspects, a “bisphosphonate moiety” refers to a molecule having two phosphate ions connected by a carbon atom (a P-C-P structure). Bisphosphonates are analogues of pyrophosphate that contain a carbon instead of an oxygen atom. The single P-C-P structure can allow a great number of possible variations, especially by changing the two lateral chains on the carbon atom. In some aspects, a “bisphosphonate moiety” exhibits a high affinity for exposed hydroxyapatite (HAP) calcium-phosphate mineral in the ECM of bone and will preferentially bind newly resorbed bone surfaces. Some bisphosphonates, such as etidronate and methylhydroxyl diphosphonate, do not inhibit the function of osteoclasts. In general, nitrogen-containing bisphosphonates inhibit osteoclasts, whereas non-nitrogen containing bisphosophonates do not inhibit osteoclasts. See U.S. Pat. No. 11,400,104 and Hokugo A et al. Bone. 2019 June; 123:115-128. In some aspects, the BP moiety does not fully or completely inhibit or only minimally inhibits osteoclast function. In some aspects, the BP only minimally inhibits farnesyl pyrophosphate synthestase (FPSS). In some aspects, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

### 2. Compositions

Disclosed herein are compositions comprising molecules, wherein the molecules comprise i) a bioactive moiety, ii) a cleavable linker, and iii) a bisphosphonate moiety, wherein the BP moiety has a P-C-P structure and wherein the cleavable linker links the bioactive moiety to the BP moiety.

i. Bioactive Moiety

In some aspects, the bioactive moiety is an osteochondral factor, an osteogenic factor, a tenogenic factor or an Extracellular Matrix (ECM) protein. In some aspects, the ECM protein is a collagen, elastin, fibrin, fibronectin, gelatin, laminin, integrin, CCN1, WISP-1 or CTGF.

In some aspects, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects, the osteochondral factor is NELL-1 or BMP-2.

In some aspects, the bioactive moiety is an osteogenic factor and the osteogenic factor is oxy133 or oxysterol-133.

In some aspects, the bioactive moiety is a tenogenic factor and the tenogenic factor is TGFβ-1, TGFβ-2, TGF-β, FGF, CTGF, BMP-12, BMP-13, BMP-14, CCN1 or WISP-1. In some aspects, the tenogenic factor is TGFβ-1 or TGFβ-2.

In some aspects, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects, the cleavable linker is a cathepsin-K-sensitive peptide linker. In some aspects, the cathepsin-K-sensitive peptide linker is GHPGGPQG (SEQ ID NO: 1) or GGGMGPSGPWGGK (SEQ ID NO: 2).

In some aspects, the cleavable linker is an MMP cleavable linker. In some aspects, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects, the BP moiety does not fully or completely inhibit or only minimally inhibits osteoclast function. In some aspects, the BP only minimally inhibits farnesyl pyrophosphate synthestase (FPSS). In some aspects, the BP moiety is 2-(pyridin-4-yl)ethane-1,1-diylbisphosphonic acid (p-PyrEBP). 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

### 3. Pharmaceutical Compositions

Disclosed herein are pharmaceutical compositions comprising molecules, wherein the molecules comprise i) a bioactive moiety, ii) a cleavable linker, and iii) a bisphosphonate moiety, wherein the BP moiety has a P-C-P structure and wherein the cleavable linker links the bioactive moiety to the BP moiety.

i. Bioactive Moiety

In some aspects, the bioactive moiety is an osteochondral factor, an osteogenic factor, a tenogenic factor or an Extracellular Matrix (ECM) protein. In some aspects, the ECM protein is a collagen, elastin, fibrin, fibronectin, gelatin, laminin, integrin, CCN1, WISP-1 or CTGF.

In some aspects, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects, the osteochondral factor is NELL-1 or BMP-2.

In some aspects, the bioactive moiety is an osteogenic factor and the osteogenic factor is oxy133 or oxysterol-133.

In some aspects, the bioactive moiety is a tenogenic factor and the tenogenic factor is TGFβ-1, TGFβ-2, TGF-β, FGF, CTGF, BMP-12, BMP-13, BMP-14, CCN1 or WISP-1. In some aspects, the tenogenic factor is TGFβ-1 or TGFβ-2.

In some aspects, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects, the cleavable linker is a cathepsin-K-sensitive peptide linker. In some aspects, the cathepsin-K-sensitive peptide linker is GHPGGPQG (SEQ ID NO: 1) or GGGMGPSGPWGGK (SEQ ID NO: 2).

In some aspects, the cleavable linker is an MMP cleavable linker. In some aspects, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects, the BP moiety does not fully or completely inhibit or only minimally inhibits osteoclast function. In some aspects, the BP only minimally inhibits farnesyl pyrophosphate synthestase (FPSS). In some aspects, the BP moiety is 2-(pyridin-4-yl)ethane-1,1-diylbisphosphonic acid (p-PyrEBP). 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the molecule, composition or pharmaceutical compositions of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### C. Methods

### 1. Methods for Targeting a Molecule to a Site of Injury

Disclosed herein are methods of targeting a molecule to a site of injury in a subject in need thereof comprising administering any one of the disclosed molecules, compositions or pharmaceutical compositions to the subject.

Disclosed herein are methods of targeting molecules to a site of injury comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of targeting a molecule to a site of injury in a subject in need thereof disclosed herein, the site of injury is enthesis tissue. In some aspects of the methods of targeting a molecule to a site of injury in a subject in need thereof disclosed herein, the site of injury a rotator cuff, a distal biceps tendon, a pectoralis major, a patellar tendon, a quadriceps tendon, or a triceps tendon.

In some aspects of the methods of targeting a molecule to a site of injury in a subject in need thereof disclosed herein, the site of injury is a site of surgery and the surgery is an ACL reconstruction, a PCL reconstruction, a LCL or MCL repair/reconstruction, a medial patellofemoral ligament reconstruction, a meniscus root repair, a meniscus allograft transplantation, or an osteochondral allograft transplantation.

In some aspects of the methods of targeting a molecule to a site of injury in a subject in need thereof disclosed herein, the molecule is administered systemically. In some aspects of the methods of targeting a molecule to a site of injury in a subject in need thereof disclosed herein, the molecule is administered locally.

i. Bioactive Moiety

In some aspects of the methods of targeting a molecule to a site of injury in a subject in need thereof disclosed herein, the bioactive moiety is an osteochondral factor, an osteogenic factor, a tenogenic factor or an Extracellular Matrix (ECM) protein. In some aspects of the methods of targeting a molecule to a site of injury in a subject in need thereof disclosed herein, the bioactive moiety is an ECM protein and the ECM protein is a collagen, elastin, fibrin, fibronectin, gelatin, laminin, integrin. CCN1, WISP-1 or CTGF.

In some aspects of the methods of targeting a molecule to a site of injury in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of targeting a molecule to a site of injury in a subject in need thereof disclosed herein, the osteochondral factor is NELL-1 or BMP-2.

In some aspects of the methods of targeting a molecule to a site of injury in a subject in need thereof disclosed herein, the bioactive moiety is an osteogenic factor and the osteogenic factor is oxy133 or oxysterol-133.

In some aspects of the methods of targeting a molecule to a site of injury in a subject in need thereof disclosed herein, the bioactive moiety is a tenogenic factor and the tenogenic factor is TGFβ-1, TGFβ-2, TGF-β3, FGF, CTGF, BMP-12, BMP-13, BMP-14, CCN1 or WISP-1. In some aspects, the tenogenic factor is TGFβ-1 or TGFβ-2.

In some aspects of the methods of targeting a molecule to a site of injury in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of targeting a molecule to a site of injury in a subject in need thereof disclosed herein, the cleavable linker is a cathepsin-K-sensitive peptide linker. In some aspects, the cathepsin-K-sensitive peptide linker is GHPGGPQG (SEQ ID NO: 1) or GGGMGPSGPWGGK (SEQ ID NO: 2).

In some aspects of the methods of targeting a molecule to a site of injury in a subject in need thereof disclosed herein, the cleavable linker is an MMP cleavable linker. In some aspects of the methods of targeting a molecule to a site of injury in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of targeting a molecule to a site of injury in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of targeting a molecule to a site of injury in a subject in need thereof disclosed herein, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate. 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

2. Methods of Treating a Tendon Injury and/or Promoting Soft Tissue to Bone Healing

Disclosed are methods of treating a tendon injury in a subject in need thereof comprising administering to the subject an effective amount of any one of the molecules, compositions or pharmaceutical compositions disclosed herein to the subject. Also disclosed are methods of promoting soft tissue to bone healing comprising administering to the subject an effective amount of any one of the molecules, compositions or pharmaceutical compositions disclosed herein to the subject.

Disclosed herein are methods of treating a tendon injury comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of treating a tendon injury in a subject in need thereof disclosed herein, the tendon injury is a rotator cuff, a distal biceps tendon, pectoralis major, patellar tendon, quadriceps tendon, or a triceps tendon.

In some aspects of the methods of treating a tendon injury in a subject in need thereof disclosed herein, the molecule is administered systemically. In some aspects of the methods of treating a tendon injury in a subject in need thereof disclosed herein, the molecule is administered locally.

i. Bioactive Moiety

In some aspects of the methods of treating a tendon injury in a subject in need thereof disclosed herein, the bioactive moiety is an osteochondral factor, an osteogenic factor, a tenogenic factor or an Extracellular Matrix (ECM) protein. In some aspects of the methods of treating a tendon injury in a subject in need thereof disclosed herein, the bioactive moiety is an ECM protein and the ECM protein is a collagen, elastin, fibrin, fibronectin, gelatin, laminin, integrin. CCN1, WISP-1 or CTGF.

In some aspects of the methods of treating a tendon injury in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of treating a tendon injury in a subject in need thereof disclosed herein, the osteochondral factor is NELL-1 or BMP-2.

In some aspects of the methods of treating a tendon injury in a subject in need thereof disclosed herein, the bioactive moiety is an osteogenic factor and the osteogenic factor is oxy133 or oxysterol-133.

In some aspects of the methods of treating a tendon injury in a subject in need thereof disclosed herein, the bioactive moiety is a tenogenic factor and the tenogenic factor is TGFβ-1, TGFβ-2, TGF-β3, FGF, CTGF, BMP-12, BMP-13, BMP-14, CCN1 or WISP-1. In some aspects of the methods of treating a tendon injury in a subject in need thereof disclosed herein, the tenogenic factor is TGFβ-1 or TGFβ-2.

In some aspects of the methods of treating a tendon injury in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK11-Fc), or activin receptor-like kinase-4-Fc (ALK4-Fc).

ii. Cleavable Linker

In some aspects of the methods of treating a tendon injury in a subject in need thereof disclosed herein, the cleavable linker is a cathepsin-K-sensitive peptide linker. In some aspects of the methods of treating a tendon injury in a subject in need thereof disclosed herein, the cathepsin-K-sensitive peptide linker is GHPGGPQG (SEQ ID NO: 1) or GGGMGPSGPWGGK (SEQ ID NO: 2).

In some aspects of the methods of treating a tendon injury in a subject in need thereof disclosed herein, the cleavable linker is an MMP cleavable linker. In some aspects of the methods of treating a tendon injury in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of treating a tendon injury in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of treating a tendon injury in a subject in need thereof disclosed herein, the BP moiety is 2-(pyridin-4-yl)ethane-1,1-diylbisphosphonic acid (p-PyrEBP). 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

### 3. Methods of Treating a Fracture

Disclosed are methods of treating a fracture in a subject in need thereof comprising administering to the subject an effective amount of any one of the molecules, compositions or pharmaceutical compositions disclosed herein to the subject. In some aspects, the fracture is a bone stress injury such as a stress reaction or a stress fracture.

Disclosed herein are methods of treating a fracture comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of treating a fracture in a subject in need thereof disclosed herein, the molecule is administered systemically. In some aspects, the molecule is administered locally.

i. Bioactive Moiety

In some aspects of the methods of treating a fracture in a subject in need thereof disclosed herein, the bioactive moiety is an osteochondral factor, an osteogenic factor, a tenogenic factor or an Extracellular Matrix (ECM) protein. In some aspects of the methods of treating a fracture in a subject in need thereof disclosed herein, the bioactive moiety is an ECM protein and the ECM protein is a collagen, elastin, fibrin, fibronectin, gelatin, laminin, integrin, CCN1, WISP-1 or CTGF.

In some aspects of the methods of treating a fracture in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of treating a fracture in a subject in need thereof disclosed herein, the osteochondral factor is NELL-1 or BMP-2.

In some aspects of the methods of treating a fracture in a subject in need thereof disclosed herein, the bioactive moiety is an osteogenic factor and the osteogenic factor is oxy133 or oxysterol-133.

In some aspects of the methods of treating a fracture in a subject in need thereof disclosed herein, the bioactive moiety is a tenogenic factor and the tenogenic factor is TGFβ-1, TGFβ-2, TGF-β, FGF, CTGF, BMP-12, BMP-13, BMP-14, CCN1 or WISP-1. In some aspects of the methods of treating a fracture in a subject in need thereof disclosed herein, the tenogenic factor is TGFβ-1 or TGFβ-2.

In some aspects of the methods of treating a fracture in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of treating a fracture in a subject in need thereof disclosed herein, the cleavable linker is a cathepsin-K-sensitive peptide linker. In some aspects of the methods of treating a fracture in a subject in need thereof disclosed herein, the cathepsin-K-sensitive peptide linker is GHPGGPQG (SEQ ID NO: 1) or GGGMGPSGPWGGK (SEQ ID NO: 2).

In some aspects of the methods of treating a fracture in a subject in need thereof disclosed herein, the cleavable linker is an MMP cleavable linker. In some aspects of the methods of treating a fracture in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of treating a fracture in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of treating a fracture in a subject in need thereof disclosed herein, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate. 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

### 4. Methods of Treating a Bone Stress Injury

Disclosed are methods of treating a bone stress injury in a subject in need thereof comprising administering to the subject an effective amount of any one of the molecules, compositions or pharmaceutical compositions disclosed herein to the subject.

Disclosed herein are methods of treating a bone stress injury comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of treating a bone stress injury in a subject in need thereof disclosed herein, the molecule is administered systemically. In some aspects of the methods of treating a bone stress injury in a subject in need thereof disclosed herein, the molecule is administered locally.

i. Bioactive Moiety

In some aspects of the methods of treating a bone stress injury in a subject in need thereof disclosed herein, the bioactive moiety is an osteochondral factor, an osteogenic factor, a tenogenic factor or an Extracellular Matrix (ECM) protein. In some aspects of the methods of treating a bone stress injury in a subject in need thereof disclosed herein, the bioactive moiety is an ECM protein and the ECM protein is a collagen, elastin, fibrin, fibronectin, gelatin, laminin, integrin, CCN1, WISP-1 or CTGF.

In some aspects of the methods of treating a bone stress injury in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of treating a bone stress injury in a subject in need thereof disclosed herein, the osteochondral factor is NELL-1 or BMP-2.

In some aspects of the methods of treating a bone stress injury in a subject in need thereof disclosed herein, the bioactive moiety is an osteogenic factor and the osteogenic factor is oxy133 or oxysterol-133.

In some aspects of the methods of treating a bone stress injury in a subject in need thereof disclosed herein, the tenogenic factor is an osteochondral factor and the tenogenic factor is TGFβ-1, TGFβ-2, TGF-β, FGF, CTGF, BMP-12, BMP-13, BMP-14, CCN1 or WISP-1. In some aspects of the methods of treating a bone stress injury in a subject in need thereof disclosed herein, the tenogenic factor is TGFβ-1 or TGFβ-2.

In some aspects of the methods of treating a bone stress injury in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of treating a bone stress injury in a subject in need thereof disclosed herein, the cleavable linker is a cathepsin-K-sensitive peptide linker. In some aspects of the methods of treating a bone stress injury in a subject in need thereof disclosed herein, the cathepsin-K-sensitive peptide linker is GHPGGPQG (SEQ ID NO: 1) or GGGMGPSGPWGGK (SEQ ID NO: 2).

In some aspects of the methods of treating a bone stress injury in a subject in need thereof disclosed herein, the cleavable linker is an MMP cleavable linker. In some aspects of the methods of treating a bone stress injury in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of treating a bone stress injury in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects, the BP moiety is 2-(pyridin-4-yl)ethane-1,1-diylbisphosphonic acid (p-PyrEBP). 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

5. Methods of Enhancing Incorporation of Allograft Tissues During or after an Orthopedic Surgical Procedure

Disclosed are methods of enhancing incorporation of allograft tissues during or after an orthopedic surgical procedure in a subject in need thereof comprising administering to the subject an effective amount of any one of the molecules, compositions or pharmaceutical compositions disclosed herein to the subject.

Disclosed herein are methods of enhancing incorporation of allograft tissues during or after an orthopedic surgical procedure comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of enhancing incorporation of allograft tissues during or after an orthopedic surgical procedure in a subject in need thereof disclosed herein, the orthopedic surgical procedure is an osteotomy, an ACL reconstruction, a PCL reconstruction, an LCL or MCL repair/reconstruction, a medial patellofemoral ligament reconstruction, a meniscus root repair, a meniscus allograft transplantation, and/or osteochondral allograft transplantation.

In some aspects of the methods of enhancing incorporation of allograft tissues during or after an orthopedic surgical procedure in a subject in need thereof disclosed herein, the orthopedic surgical procedure is osteointegration of an implantable prosthesis. In some aspects, the implantable prosthesis is a implant material, such as, but not limited to, metal, ceramic or plastic, that is implanted into bone or dental applications.

In some aspects of the methods of enhancing incorporation of allograft tissues during or after an orthopedic surgical procedure in a subject in need thereof disclosed herein, the orthopedic surgical procedure is osteointegration of allograft tissue. In some aspects, allograft tissue is osteochondral allografts and/or bone allografts.

In some aspects of the methods of enhancing incorporation of allograft tissues during or after an orthopedic surgical procedure in a subject in need thereof disclosed herein, the molecule is administered just before the orthopedic surgical procedure. In some aspects of the methods of enhancing incorporation of allograft tissues during or after an orthopedic surgical procedure in a subject in need thereof disclosed herein, the molecule is administered during the orthopedic surgical procedure. In some aspects of the methods of enhancing incorporation of allograft tissues during or after an orthopedic surgical procedure in a subject in need thereof disclosed herein, the molecule is administered after the orthopedic surgical procedure.

In some aspects of the methods of enhancing incorporation of allograft tissues during or after an orthopedic surgical procedure in a subject in need thereof disclosed herein, the molecule is administered systemically. In some aspects, the molecule is administered locally.

i. Bioactive Moiety

In some aspects of the methods of enhancing incorporation of allograft tissues during or after an orthopedic surgical procedure in a subject in need thereof disclosed herein, the bioactive moiety is an osteochondral factor, an osteogenic factor, a tenogenic factor or an Extracellular Matrix (ECM) protein. In some aspects of the methods of enhancing incorporation of allograft tissues during or after an orthopedic surgical procedure in a subject in need thereof disclosed herein, the bioactive moiety is an ECM protein and the ECM protein is a collagen, elastin, fibrin, fibronectin, gelatin, laminin, integrin, CCN1, WISP-1 or CTGF.

In some aspects of the methods of enhancing incorporation of allograft tissues during or after an orthopedic surgical procedure in a subject in need thereof disclosed herein, the bioactive moiety is an osteochondral factor and the osteochondral factor is NELL-1, BMP-1, BMP-2, TGF-1, TGF-β2, TGF-β3, BMP-3, BMP-4, BMP-5, BMP-6, BMP-7, BMP-8a, BMP-8b, BMP-10, BMP-11, BMP-15, or rhBMP. In some aspects of the methods of enhancing incorporation of allograft tissues during or after an orthopedic surgical procedure in a subject in need thereof disclosed herein, the osteochondral factor is NELL-1 or BMP-2.

In some aspects of the methods of enhancing incorporation of allograft tissues during or after an orthopedic surgical procedure in a subject in need thereof disclosed herein, the bioactive moiety is an osteogenic factor and the osteogenic factor is oxy133 or oxysterol-133.

In some aspects of the methods of enhancing incorporation of allograft tissues during or after an orthopedic surgical procedure in a subject in need thereof disclosed herein, the bioactive moiety is a tenogenic factor and the tenogenic factor is TGFβ-1, TGFβ-2, TGF-β3, FGF, CTGF, BMP-12, BMP-13, BMP-14, CCN1 or WISP-1. In some aspects of the methods of enhancing incorporation of allograft tissues during or after an orthopedic surgical procedure in a subject in need thereof disclosed herein, the tenogenic factor is TGFβ-1 or TGFβ-2.

In some aspects of the methods of enhancing incorporation of allograft tissues during or after an orthopedic surgical procedure in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of enhancing incorporation of allograft tissues during or after an orthopedic surgical procedure in a subject in need thereof disclosed herein, the cleavable linker is a cathepsin-K-sensitive peptide linker. In some aspects of the methods of enhancing incorporation of allograft tissues during or after an orthopedic surgical procedure in a subject in need thereof disclosed herein, the cathepsin-K-sensitive peptide linker is GHPGGPQG (SEQ ID NO: 1) or GGGMGPSGPWGGK (SEQ ID NO: 2).

In some aspects of the methods of enhancing incorporation of allograft tissues during or after an orthopedic surgical procedure in a subject in need thereof disclosed herein, the cleavable linker is an MMP cleavable linker. In some aspects of the methods of enhancing incorporation of allograft tissues during or after an orthopedic surgical procedure in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of enhancing incorporation of allograft tissues during or after an orthopedic surgical procedure in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of enhancing incorporation of allograft tissues during or after an orthopedic surgical procedure in a subject in need thereof disclosed herein, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

6. Methods of Promoting Repair or Regeneration of Enthesis During or after an Orthopedic Surgical Procedure

Disclosed are methods of promoting repair or regeneration regeneration of enthesis during or after an orthopedic surgical procedure in a subject in need thereof comprising administering to the subject an effective amount of any one of the molecules, compositions or pharmaceutical compositions disclosed herein to the subject.

Disclosed herein are methods of promoting repair or regeneration of enthesis during or after an orthopedic surgical procedure comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of promoting repair or regeneration of enthesis during or after an orthopedic surgical procedure in a subject in need thereof disclosed herein, the orthopedic surgical procedure is an osteotomy, an ACL reconstruction, a PCL reconstruction, an LCL or MCL repair/reconstruction, a medial patellofemoral ligament reconstruction, a meniscus root repair, a meniscus allograft transplantation, and/or osteochondral allograft transplantation.

In some aspects of the methods of promoting repair or regeneration of enthesis during or after an orthopedic surgical procedure in a subject in need thereof disclosed herein, the molecule is administered just before the orthopedic surgical procedure. In some aspects of the methods of promoting repair or regeneration of enthesis during or after an orthopedic surgical procedure in a subject in need thereof disclosed herein, the molecule is administered during the orthopedic surgical procedure. In some aspects of the methods of promoting repair or regeneration of enthesis during or after an orthopedic surgical procedure in a subject in need thereof disclosed herein, the molecule is administered after the orthopedic surgical procedure.

In some aspects of the methods of promoting repair or regeneration of enthesis during or after an orthopedic surgical procedure in a subject in need thereof disclosed herein, the molecule is administered systemically. In some aspects, the molecule is administered locally.

i. Bioactive Moiety

In some aspects of the methods of promoting repair or regeneration of enthesis during or after an orthopedic surgical procedure in a subject in need thereof disclosed herein, the bioactive moiety is an osteochondral factor, an osteogenic factor, a tenogenic factor or an Extracellular Matrix (ECM) protein. In some aspects of the methods of promoting repair or regeneration of enthesis during or after an orthopedic surgical procedure in a subject in need thereof disclosed herein, the bioactive moiety is an ECM protein and the ECM protein is a collagen, elastin, fibrin, fibronectin, gelatin, laminin, integrin, CCN1, WISP-1 or CTGF.

In some aspects of the methods of promoting repair or regeneration of enthesis during or after an orthopedic surgical procedure in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of promoting repair or regeneration of enthesis during or after an orthopedic surgical procedure in a subject in need thereof disclosed herein, the osteochondral factor is NELL-1 or BMP-2.

In some aspects of the methods of promoting repair or regeneration of enthesis during or after an orthopedic surgical procedure in a subject in need thereof disclosed herein, the bioactive moiety is an osteogenic factor and the osteogenic factor is oxy133 or oxysterol-133.

In some aspects of the methods of promoting repair or regeneration of enthesis during or after an orthopedic surgical procedure in a subject in need thereof disclosed herein, the bioactive moiety is a tenogenic factor and the tenogenic factor is TGFβ-1, TGFβ-2, TGF-β3, FGF, CTGF, BMP-12, BMP-13, BMP-14, CCN1 or WISP-1. In some aspects of the methods of promoting repair or regeneration of enthesis during or after an orthopedic surgical procedure in a subject in need thereof disclosed herein, the tenogenic factor is TGFβ-1 or TGFβ-2.

In some aspects of the methods of promoting repair or regeneration of enthesis during or after an orthopedic surgical procedure in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of promoting repair or regeneration of enthesis during or after an orthopedic surgical procedure in a subject in need thereof disclosed herein, the cleavable linker is a cathepsin-K-sensitive peptide linker. In some aspects of the methods of promoting repair or regeneration of enthesis during or after an orthopedic surgical procedure in a subject in need thereof disclosed herein, the cathepsin-K-sensitive peptide linker is GHPGGPQG (SEQ ID NO: 1) or GGGMGPSGPWGGK (SEQ ID NO: 2).

In some aspects of the methods of promoting repair or regeneration of enthesis during or after an orthopedic surgical procedure in a subject in need thereof disclosed herein, the cleavable linker is an MMP cleavable linker. In some aspects of the methods of promoting repair or regeneration of enthesis during or after an orthopedic surgical procedure in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of promoting repair or regeneration of enthesis during or after an orthopedic surgical procedure in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of promoting repair or regeneration of enthesis during or after an orthopedic surgical procedure in a subject in need thereof disclosed herein, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

7. Methods of Treating Osteoporosis, Osteoarthritis and/or Cartilage Defects

Disclosed herein are methods of treating osteoporosis, osteoarthritis and/or cartilage defects in a subject in need thereof comprising administering any one of the disclosed molecules, compositions or pharmaceutical compositions to the subject.

Disclosed herein are methods of methods of treating osteoporosis, osteoarthritis and/or cartilage defects in a subject in need thereof comprising administering molecules comprising: i) a bioactive moiety, ii) a cleavable linker, and iii) a bisphosphonate moiety, wherein the BP moiety has a P-C-P structure, and wherein the cleavable linker links the bioactive moiety to the BP moiety.

In some aspects of the methods of treating osteoporosis, osteoarthritis and/or cartilage defects in a subject in need thereof disclosed herein, the site of osteoporosis, osteoarthritis and/or cartilage defects is the knee, hip, ankle spine, wrist, shoulder, elbow, hands, fingers, feet and/or toes.

In some aspects of the methods of treating osteoporosis, osteoarthritis and/or cartilage defects in a subject in need thereof disclosed herein, the molecule is administered systemically. In some aspects of the methods of treating osteoporosis, osteoarthritis and/or cartilage defects in a subject in need thereof disclosed herein, the molecule is administered locally.

i. Bioactive Moiety

In some aspects of the methods of treating osteoporosis, osteoarthritis and/or cartilage defects in a subject in need thereof disclosed herein, the bioactive moiety is an osteochondral factor, an osteogenic factor, a tenogenic factor or an Extracellular Matrix (ECM) protein. In some aspects of the methods of treating osteoporosis, osteoarthritis and/or cartilage defects in a subject in need thereof disclosed herein, the bioactive moiety is an ECM protein and the ECM protein is a collagen, elastin, fibrin, fibronectin, gelatin, laminin, integrin, CCN1, WISP-1 or CTGF.

In some aspects of the methods of treating osteoporosis, osteoarthritis and/or cartilage defects in a subject in need thereof disclosed herein, the bioactive moiety is an osteochondral factor and the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP. In some aspects of the methods of treating osteoporosis, osteoarthritis and/or cartilage defects in a subject in need thereof disclosed herein, the osteochondral factor is NELL-1 or BMP-2.

In some aspects of the methods of treating osteoporosis, osteoarthritis and/or cartilage defects in a subject in need thereof disclosed herein, the bioactive moiety is an osteogenic factor and the osteogenic factor is oxy133 or oxysterol-133.

In some aspects of the methods of treating osteoporosis, osteoarthritis and/or cartilage defects in a subject in need thereof disclosed herein, the bioactive moiety is a tenogenic factor and the tenogenic factor is TGFβ-1, TGFβ-2, TGF-β, FGF, CTGF, BMP-12, BMP-13, BMP-14. CCN1 or WISP-1. In some aspects, the tenogenic factor is TGFβ-1 or TGFβ-2.

In some aspects of the methods of treating osteoporosis, osteoarthritis and/or cartilage defects in a subject in need thereof disclosed herein, the bioactive moiety is the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

ii. Cleavable Linker

In some aspects of the methods of treating osteoporosis, osteoarthritis and/or cartilage defects in a subject in need thereof disclosed herein, the cleavable linker is a cathepsin-K-sensitive peptide linker. In some aspects, the cathepsin-K-sensitive peptide linker is GHPGGPQG (SEQ ID NO: 1) or GGGMGPSGPWGGK (SEQ ID NO: 2).

In some aspects of the methods of treating osteoporosis, osteoarthritis and/or cartilage defects in a subject in need thereof disclosed herein, the cleavable linker is an MMP cleavable linker. In some aspects of the methods of treating osteoporosis, osteoarthritis and/or cartilage defects in a subject in need thereof disclosed herein, the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

In some aspects, the cleavable linker is a pH sensitive linker. pH sensitive linkers can be those found in U.S. Pat. No. 8,063,209. U.S. Pat. Nos. 11,219,697 and 11,840,549, all of which are hereby incorporated by reference in their entireties. pH sensitive linkers can also be found, for example, in U.S. Pat. No. 10,383,912, 10,688,193 both of which are hereby incorporated by reference in their entireties.

iii. Bisphosphonate Moiety

In some aspects of the methods of treating osteoporosis, osteoarthritis and/or cartilage defects in a subject in need thereof disclosed herein, the BP moiety does not fully or completely inhibit or only minimally inhibits osteoclast function. In some aspects, the BP only minimally inhibits farnesyl pyrophosphate synthestase (FPSS). In some aspects of the methods of treating osteoporosis, osteoarthritis and/or cartilage defects in a subject in need thereof disclosed herein, the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

### 8. Dosing Regimens

Disclosed are dosing regimens comprising at least one treatment cycle of an effective amount of any of the disclosed molecules, compositions or pharmaceutical compositions.

Disclosed herein is are dosing regimens in which the molecule, composition or pharmaceutical composition is administered only once.

Disclosed herein are dosing regimens in which the molecule, composition or pharmaceutical composition is administered multiple times over a period of time.

Disclosed herein are dosage regiments in which administration the molecule, composition or pharmaceutical composition can occur anywhere from 1 day to 2 weeks after surgery.

Treatment cycles can include the administration of different dosages of molecules, compositions or pharmaceutical compositions as well as administration at different time points. The molecule, compositions or pharmaceutical compositions can be administered for varying amounts of time for up to 6 months. The molecules, compositions or pharmaceutical compositions can be administered for varying amounts of time indefinitely. In some instances, the administration can occur for up to one, two, three, four, five or six months. For example, the molecule, composition or pharmaceutical composition can be administered once a week for 4, 6, 8, 10, 12, 14, 16, 18, 20, 22, or 24 weeks. In some aspects the disclosed molecules, compositions or pharmaceutical compositions can be administered about every 3, about every 6 or about every 12 months.

The length of time for each treatment cycle can vary depending on the amount of molecule, composition or pharmaceutical composition administered per dosage. A treatment cycle can include the administration of a molecule, composition or pharmaceutical composition once, twice or three times a week. In some aspects, the molecule, composition or pharmaceutical composition can be administered daily. In some aspects, the molecule, composition or pharmaceutical composition can be administered once every two weeks or even once a month. In some instances, the molecule, composition or pharmaceutical composition can be administered every two weeks for 4, 6, 8, 10, 12, 14, 16, 18, 20, 22, or 24 weeks. For example, the treatment cycle can include administering a molecule, composition or pharmaceutical composition once a week for four weeks or once every two weeks for up to six months. Thus, each treatment cycle includes an established length of time for administration as well as an established dosing schedule during that time frame.

In one aspect, more than molecule, composition or pharmaceutical composition can be administered during the treatment cycles. The more than one molecule, composition or pharmaceutical composition can be formulated together or in separate compositions. In some instances, one or more molecules, compositions or pharmaceutical compositions is administered in combination with one or more other therapeutic agents, including, but not limited to antibodies, nanobodies, aptamers, liposomes, antioxidants, anti-inflammatory agents, senolytic agents.

### 9. Dose

The dose or dosage of molecule, composition or pharmaceutical composition can vary depending on many factors, such as but not limited to, age, condition, sex and extent of the disease in the patient, route of administration, length of treatment cycle, or whether other drugs are included in the regimen, and can be determined by one of skill in the art.

Effective dosages can be determined empirically, and making such determinations is within the skill in the art. The dosage ranges for the administration of the compositions are those large enough to produce the desired effect in which the disease is treated. For example, the dosage can be an amount effective to provide therapeutic effects and provide or allow for sustained therapeutic effects even after the treatment (e.g. one or more of the disclosed molecules comprising: i) a bioactive moiety, ii) a cleavable linker, and iii) a bisphosphonate moiety, wherein the BP moiety has a P-C-P structure and wherein the cleavable linker links the bioactive moiety to the BP moiety) is withdrawn. The therapeutic effects can be, but are not limited to, an improvement in a tendon injury, the healing of a fracture or bone stress injury, a faster recovery from an orthopedic procedure, improved biomechanical properties, such as strength, or improved tissue organization of the repaired enthesis compared to a subject who did not receive the treatment. Other biomarkers used to measure therapeutic effects can be markers of tendon or bone repair. The therapeutic effects can be measured by imaging techniques, including MRI, intravascular ultrasound, ultrafast imaging CT scans, B-mode ultrasonography, virtual histology intravascular ultrasound, optical coherence tomography, or other known methods.

The dosage should not be so large as to cause adverse side effects, such as unwanted cross-reactions, anaphylactic reactions, and the like. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

Suitable dosages include, but are not limited to amounts between 0.01 mg/kg and 20 mg/kg. For example, disclosed herein are methods involving administering one or more of the disclosed molecules, compositions or pharmaceutical compositions to a subject, wherein the molecule is administered in an amount of about 0.01 mg/kg to about 20 mg/kg. For example, the concentration of the molecule can be 0.01, 0.1, 1, 2, 3, 4, 5, 6, 7, 8, 9, 10, 11, 12, 13, 14, 15, 16, 17, 18, 19, or 20 mg/kg. Doses will depend on the identity of the bioactive moiety. Doses of BP-BMP-2, for example, may be about 10 mg/kg.

The molecule, composition or pharmaceutical composition dose can be administered systemically or locally. The molecule, composition or pharmaceutical composition dose can be administered as a bolus injection or as an infusion over one or more hours.

### 10. Delivery

In the methods described herein, administration or delivery of the molecules, compositions or pharmaceutical compositions can be via a variety of mechanisms. As defined above, disclosed herein are methods of treating, dosing regimens and methods of using those dosing regimens to treat. The dosing regimens and methods include compositions containing any one or more of the molecules described herein that can also include a carrier such as a pharmaceutically acceptable carrier. For example, disclosed are pharmaceutical compositions, comprising the molecules and compositions disclosed herein, and a pharmaceutically acceptable carrier.

The disclosed molecules, compositions or pharmaceutical compositions can be in solution or in suspension (for example, incorporated into microparticles, liposomes, or cells).

Any suitable route of administration can be used for the disclosed molecules, compositions and pharmaceutical compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavemosal, intramedullar, intraocular, intracranial, transdermal, transmucosal, transnasal, inhalational, intracisternal, epidural, peridural, intravitreal, etc. The disclosed compositions can be used in and with any other therapy.

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

### 11. Combination Therapy

In one aspect of the disclosed methods, molecules, compositions or pharmaceutical compositions can be administered alone or in combination with one or more additional therapeutic agents. The additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005). For example, pharmaceutical compositions containing molecules can be administered in combination with one or more known therapeutic agents for treating atherosclerosis.

Examples of therapeutic agents that treat tendon injuries include, but are not limited to, anti-inflammatory agents, analgesic agents, anti-rheumatologic agents, and immune modulating agents, biophysical agents (e.g. shock wave therapy, ultrasound, magnetic fields, joint and tissue passive motion devices), dry needling, thermal therapy (e.g ice, heat) and therapeutic exercises (e.g. physical therapy, eccentric strengthening).

The molecules, compositions or pharmaceutical compositions can be administered in conjunction with or followed by any of the disclosed additional therapeutics.

The combination therapies can include administering the molecule, composition or pharmaceutical composition and an additional therapeutic agent during the treatment cycle of a dosing regimen.

### EXAMPLES

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

### Example 1: Biologically-Coupled Bisphosphonate Chaperones Effectively Deliver Molecules to the Site of Soft Tissue-Bone Healing

Tendon injuries are common and often treated surgically, however, current tendon repair healing results in poorly organized fibrotic tissue. While certain growth factors have been reported to improve both the strength and organization of the repaired enthesis, their clinical applicability is severely limited due to a lack of appropriate delivery strategies. In this study, a recently developed fluorescent probe, Osteoadsorptive Fluorogenic Sentinel-3 (OFS-3) that is composed of a bone-targeting bisphosphonate moiety linked to fluorochrome and quencher molecules joined via a cathepsin K-sensitive peptide sequence was evaluated. Using a murine Achilles tendon-to-bone repair model, bisphosphonate-based and/or Ctsk-coupled imaging probes were applied either locally or systemically. Fluorescence imaging was used to quantify the resultant signal in vivo. After tendon-bone repair, animals that received either local or systemic administration of imaging probes demonstrated significantly higher fluorescence signal at the repair site compared to the sham surgery group at all time points (p<0.001), with signal peaking at 7 to 10 days after surgery. The findings demonstrate the feasibility of using a novel bisphosphonate-based targeting and Ctsk-activated delivery of molecules to the site of tendon-to-bone repair and creates a foundation for further development of this platform as an effective strategy to deliver bioactive molecules to sites of musculoskeletal injury.

Bisphosphonates (BPs) are molecules that have an intrinsic affinity for exposed hydroxyapatite (HAP) calcium-phosphate mineral in the ECM of bone and will preferentially bind newly resorbed bone surfaces. HAP mineral can be exposed in bone by mechanical disruption such as the decortication of bone at the time of tendon repair or by osteoclasts activated physiologically during times of increased bone metabolic activity. At the time of surgical repair of tendon to bone, mechanically disrupted local bone at a tendon footprint exposes HAP and creates an environment where local bone-derived progenitor cells are in direct communication with the repaired tendon. In addition to mesenchymal progenitor cells, osteoclasts are also known to be active at sites of tendon repair and have been linked to the bone loss associated with tendon repairs to bone¹⁶⁻¹⁹. Given that activated osteoclasts secrete catabolic bone enzymes like cathepsin K (Ctsk) during the repair process, it is possible to take advantage of this well-characterized biology to facilitate targeted delivery of molecules to the site of tendon-bone repair.

### Methods

### Murine Model of Tendon-Bone Repair

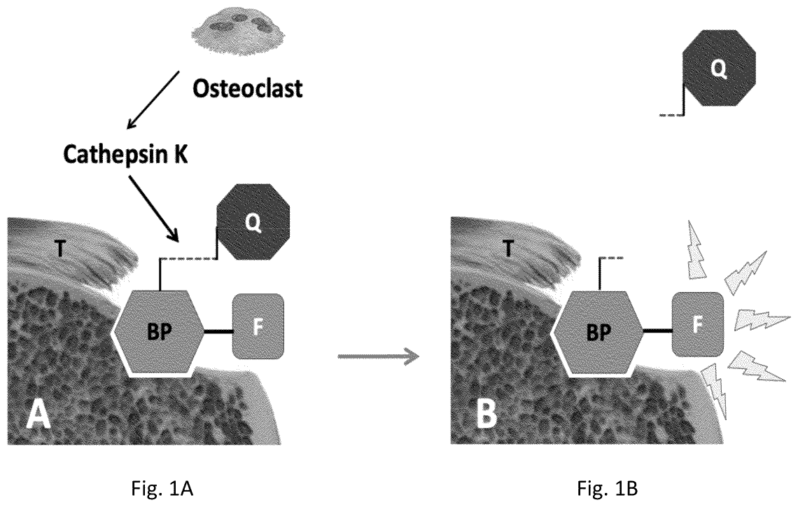

12-week-old male C57BL/6 mice underwent right hindlimb surgery for either Achilles tendon-to-bone repair or sham surgery. Achilles tendon-to-bone repair consisted of a transtendinous 5-0 nylon suture placed orthogonal to the long axis of the Achilles tendon as well as a transosseous suture placed at the posterior calcaneus placed from lateral to medial. After appropriate suture capture the Achilles tendon was sharply transected from its attachment at the calcaneus followed by decortication of posterior-superior portion of the calcaneus with a dental burr. The Achilles tendon was then repaired to bone via direct apposition of the transected tendon proximal end to the decorticated region of the calcaneus using the previously placed 5-0 nylon suture (FIG. 1). The operative hindlimbs were not immobilized and animals were weight bearing as tolerated immediately after the procedure. At the time of necropsy, rupture of the repair construct was rare and affected 5% or less of the study animals.

### In Vivo Imaging

AF647-ZOL, a commercially available imaging probe composed of a BP conjugated to fluorochrome (Alexa Fluor 647) was obtained from BioVinc, LLC (Pasadena, CA). OFS-3 was synthesized as previously described.²⁰ In brief OFS-3 is composed of a BP molecule conjugated to the fluorochrome sulfo-cyanine 5 as well as the Ctsk-sensitive peptide sequence GHPGGPQG. The other end of this peptide sequence is conjugated to BlackBerry Quencher 650 (BBQ-650), a dye which internally absorbs the spectrum of light emission from the properly excited Sulfo-Cyanine 5 fluorochrome via a Forster Resonance Energy Transmission (FRET) mechanism. Cat K 680 FAST (Perkin-Elmer, Inc) is a commercially available in vivo imaging agent that has no BP component, but is composed of a similar fluorochrome-quencher dye pair connected via a Ctsk-sensitive peptide. When activated by Ctsk the Cat K 680 FAST probe will also generate fluorescence when exposed to light energy at the appropriate excitation spectrum, however, it will not bind specifically to bone. Each study drug was resuspended in sterile phosphate buffered saline (PBS) at pH 7.4.

Animals in the local treatment groups (n=7 per group) underwent Achilles tendon-bone repair followed by a single dose of 5 microliters (μL) of 1000 nM solution of study drug (AF647-ZOL or OFS-3) administered manually via micro-pipet directly onto the repaired tendon-bone junction before skin closure. The local treatment group does not include Cat K 680 FAST because this product has no bone-localizing bisphosphonate component. Similarly, local OSF-3 was not included in the sham group because these animals only have a skin injury; they have no mechanically exposed bone matrix containing hydroxyapatite minerals and/or any evidence of activation of local resident osteoclasts. Thus OSF-3 is not expected to exhibit significant differential targeting among sham animals.

Animals in the systemic treatment groups (n=7 per group for OFS-3 and AF647; n=3 per group for Cat K 680 FAST) underwent Achilles tendon-bone repair followed by a single dose of either 100 μL of 50 nM study drug (AF647-ZOL or OFS-3) or 2 nmol in 100 μL of Cat K 680 FAST via subcutaneous injection immediately following surgery. The sham surgery groups (n=7 per group) in each experiment received a skin incision and suture closure of the skin without disruption of the Achilles tendon or calcaneus followed by systemic injection of either 100 μL of 50 nM study drug (AF647-ZOL or OFS-3) or 2 nmol in 100 μL of Cat K 680 FAST.

Treatment groups were designed to compare the current gold standard clinical approach (local application) to the potential most challenging scenario, future translational approach (systemic administration). Currently, the only clinically available growth factor for the treatment of orthopaedic injuries (BMP-2) is applied topically to the site of repair at the time of surgery. Delivery of the study compound will likely be to the site of repair via percutaneous injection. In rodents a portion of any percutaneous injection of investigational compound is likely delivered to regions adjacent to the anatomic target in addition to the anatomic target itself. As a result, evaluating the localization of the imaging compounds in the most challenging administration scenario (i.e. distant from the Achilles tendon-calcaneus repair site) provides data regarding the targeting ability of the investigational compound.

Fluorescent signal (p/s) was obtained while the animals were under isoflurane anesthesia using the IVIS Lumina II imaging system (Perkin-Elmer Inc., Shelton, CT) on post-operative day (POD) 1 and then every 72 hours for either 3 weeks (AF647-ZOL and OFS-3 treatment groups) or t 1 days (Cat K 680 FAST treatment groups). Fluorescent signal from the region of interest at the operative limb hindfoot (sham and repair groups) and contralateral non-operative hindlimb was quantified using IVIS Living Image® advanced in vivo imaging software (Perkin-Elmer Inc., Shelton, CT). The hindfoot of the operative hindlimb was designated as “on-target” among animals who underwent tendon repair and this was noted to have the highest fluorescence signal throughout the course of the experiments. The forefoot of the non-operative hindlimb was designated as “off-target” among all of the animals as this region was noted to have the second highest fluorescence signal throughout the course of the experiments. For animals in the sham treatment groups the forefoot of the operative hindlimb was designated as “on-target” as this region was closest to the surgical site and the forefoot was noted to have higher fluorescence signal that the hindfoot throughout the course of the experiments.

### Histology and Immunohistochemistry

Four days after Achilles transection and repair, mouse hindlimb samples were harvested and subsequently fixed in 4% Paraformaldehyde for 3 days. Samples were then decalcified using formic acid (Statlab, Cat: 1414-32) for 2 days and embedded in paraffin. Sagittal sections 5 micrometers in thickness were acquired using a microtome and stained with Hematoxylin (Fisher brand, Cat: 245-656) and Eosin (Sigma-Aldrich, Cat: 17372-87-1) (H&E) and counterstained with Safranin-O (Sigma-Aldrich, Cat: 58884-25G) and Fast Green (Sigma-Aldrich, Cat: F7258-25G) (SOFG), using a standard histology protocol as previously described²¹. Evaluation of activated osteoclasts was performed with immunohistochemistry staining of sections for cathepsin K (Abcam, Cat: ab19027) at a dilution of 1:100 using standard protocols used as previously described²². Images were taken with the Olympus BX60 brightfield microscope using a 4× and 10× objective lens. Representative sections throughout the posterior calcaneal-enthesis region were chosen for histologic analysis.

### Statistical Analysis

Mean fluorescence signal was compared across groups and between “on-” versus “off-target” using a repeated measure (mixed) analysis of variance model. The repeated measures model was needed since the same animals are measured across time, making these observations non-independent. Examination of residual error normal quantile plots confirmed that the data has a normal distribution on the log scale, thus allowing use of this parametric model.

### Results

In Vivo Imaging with AF647-ZOL

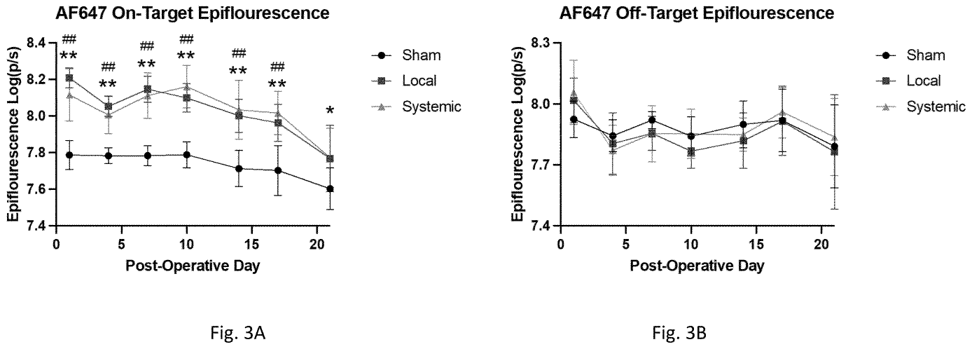

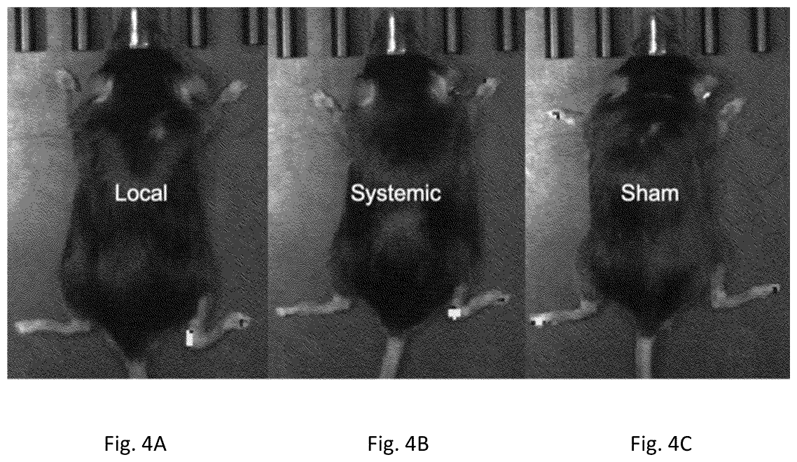

Animals in repair cohorts that received either local or systemic AF647-ZOL demonstrated significantly higher on-target fluorescence signal compared to the sham group at all time points (p<0.002), except for POD 21 (p=0.032), with signal peaking at 7 to 10 days after surgery (FIG. 3). Representative images from each treatment group on POD 10 are included in FIG. 4. There was no significant difference in on-target signal between the local and systemic treatment groups at all time points studied. Fluorescence at the site of repair (on-target signal) in the repaired (both local and systemically treated) cohorts was significantly higher than off-target fluorescence in sham and repaired cohorts (p<0.001) except for POD 1 in the systemic AF647-ZOL treatment group (p=0.31) and on POD 17 and 21 in both the systemic and local treatment groups. This likely reflects the level of signal returning to baseline at approximately POD 17 or ~2 weeks after surgery.

In Vivo Imaging with Cat K 680 FAST

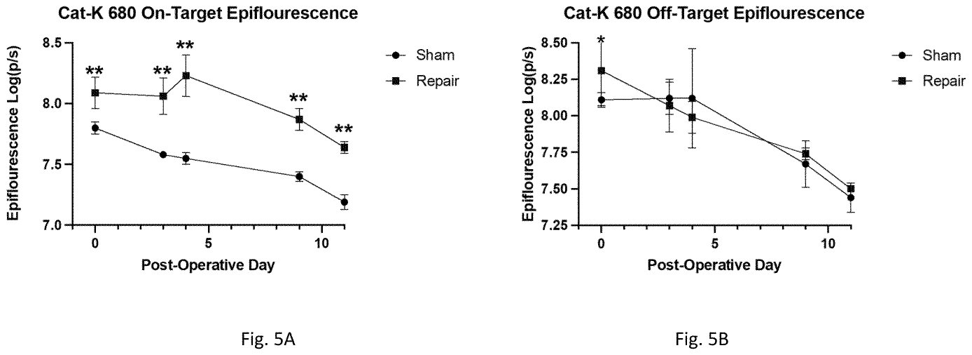

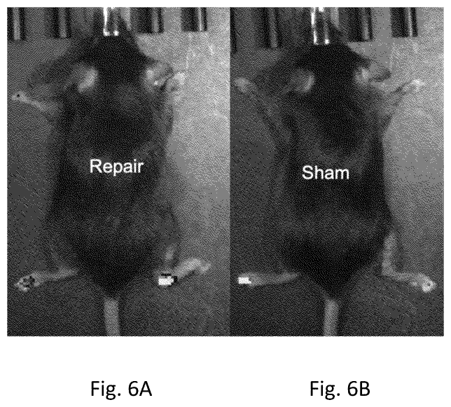

Animals in the repair group that received Cat K 680 FAST demonstrated significantly higher on-target fluorescence signal compared to the sham group at all time points (p<0.0001) except on POD 0 (p=0.0065, FIG. 5). Fluorescence signal in the repair cohorts peaked at approximately 4 days after surgery. Off-target fluorescence signal in the repair group compared to sham was significantly different on POD 0 (p=0.048), however the remainder of the time points studied were not significantly different. Repair group animals demonstrated on-target fluorescence signal that was significantly less than off-target signal on POD 0 (p=0.033), yet significantly greater than off-target signal on POD 4 (p=0.022). However, the sham group animals demonstrated significantly increased off-target signal relative to on-target signal at all time points (p<0.014). Representative images from each treatment group on POD 4 are included in FIG. 6.

In Vivo Imaging with OFS-3

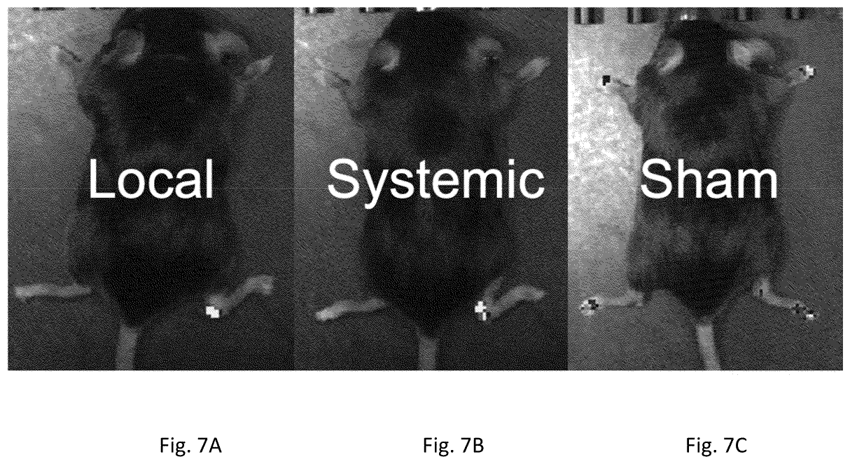

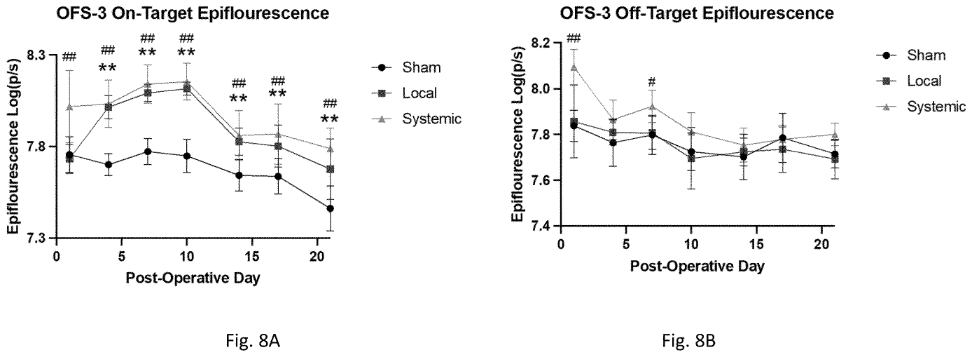

Animals in repair cohorts that received either local or systemic OFS-3 demonstrated significantly higher on-target fluorescence signal compared to the sham group at all time points (p<0.001) except for the repair with local OFS-3 treatment group at POD 1 (P=0.66, FIG. 7). Signal in the repair cohorts peaked at 7 to 10 days after surgery. Of note, there was a significant difference in on-target signal between the local and systemic treatment groups on POD 1 (p=0.0001). However, there was no significant difference in on-target signal between the local and systemic treatment groups after POD 1. On-target fluorescence signal in the repair cohorts was significantly higher than the off-target fluorescence in either the sham or repaired cohorts (p<0.001) from POD 4 to 14, however, the repair with systemic OFS-3 treatment group was not significantly different on POD 1 (p<0.14, FIG. 8). After POD 14, there was no significant difference in on-target signal compared to off-target signal in the repair cohorts (FIG. 8). Similar to the AF647-ZOL results, this likely reflects the level of signal returning to baseline at approximately 2 weeks after surgery.

### Histology and Immunohistochemistry

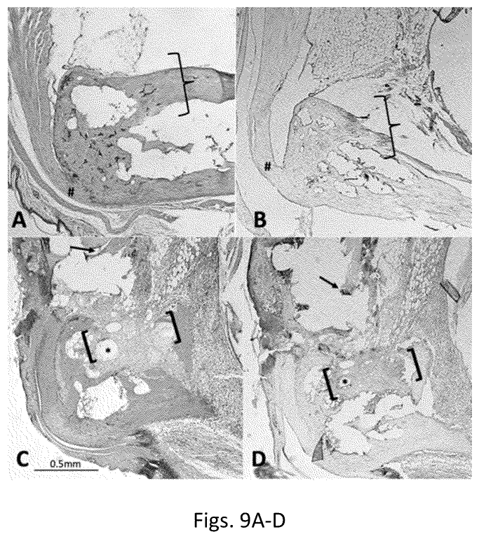

Uninjured contralateral hindlimbs (FIG. 9A, B) demonstrated normal bone, tendon and enthesis architecture on H&E and SOFG staining and a distinct lack of significant cathepsin-K positive cells at the posterior-superior calcaneus. The sections were decalcified to enable sectioning through bone. This precluded direct localization of OSF-3 or AF647 because for bisphosphonate molecules (BP) to associate with bone matrix, divalent calcium cations must be available to interact within the hydroxyapatite minerals to which BP molecules bind. However, Ctsk activity was examined. Animals treated with Achilles tendon-to-bone repair (FIG. 9C and FIG. 9D) demonstrated a decorticated region of the posterior-superior calcaneus with significant Ctsk-positive staining on immunohistochemistry at this area of decorticated bone (black brackets, FIG. 9D). Additional areas of Ctsk-positive cells were present at the adjacent skin tissue as well as a small degree of staining at the residual distal Achilles tendon stump (black arrow, FIG. 9D).

### Discussion

Currently the targeting of bioactive molecules like BMP-2 to the site of tendon-bone repair in the clinical setting is limited to supraphysiologic doses administered at the gross tissue level and only at the time of the surgical procedure²¹. BP-based compounds can effectively target the site of tendon-bone repair in vivo. Furthermore, the biologically-coupled Ctsk dependent molecular delivery strategy can effectively target the site of tendon-bone repair whether delivered locally or systemically.

This study evaluated the BP moiety as a means of targeting active bone remodeling in the context of Achilles tendon repair, not as a therapeutic drug. The use of BP molecules to augment soft tissue-to-bone healing has been investigated previously. However, this has largely been in the context of animal models investigating the effects osteoclast-inhibiting BPs and the results of this approach have been controversial. Xu et al. reported that risedronate was beneficial to rotator cuff healing in among osteoporotic rats²⁴. Liu et al. noted enhanced tendon graft-to-bone healing in a rat model of ACL reconstruction²⁵. In contrast, Hjorthaug et al. noted negative effects on tendon-to-bone healing in their study of zolendronate on the healing of rat rotator cuff repairs²⁶. In their model of canine tendon-to-bone healing, Thomopoulos et al. demonstrated improved ultimate load to failure and a lower incidence of suture pull through tendon after treatment with alendronate²⁷. Of note, all of these studies are of very limited relevance to the current study as the BPs included in these studies are osteoclast-inhibiting and not biologically-coupled to other molecules as is the case with OFS-3. Sung et al. demonstrated that low dose alendronate had little effect on the viability, proliferation and wound healing capacity of human rotator cuff tendon fibroblasts in vitro²⁸. Finally, although there has been some potential risk of increased tendon injury reported to be associated with the clinical use of BPs, it is difficult to attribute this risk to the use of BPs alone²⁹. In addition, given the widespread use of bisphosphonate both therapeutically (e.g. osteoporosis)³⁰ and diagnostically (e.g. bone scintigraphy)³¹, the transient use of these agents appears safe. Furthermore, it is possible to engineer bisphosphonate molecules that bind HAP without significantly impairing local osteoclast activity^{32;33} as is the case with OFS-3.

Bone loss at the site of tendon repair is a well-described phenomenon and this bone loss has been noted to be associated with presence of activated osteoclasts¹⁶⁻¹⁹. The results corroborate this finding as evidenced by the histologic findings demonstrating significant cathepsin K positivity on immunohistochemistry staining of repaired samples. Interestingly, there was some Ctsk positivity at the edges of the transected tendon in addition to the more pronounced Ctsk positive staining at the site of tendon repair to bone. The presence of Ctsk on POD 4 and POD 7 immunohistochemistry-stained sections correlates well with in vivo imaging data from POD 4 and POD 7. Local activated osteoclast biology has provided the predictable biodistribution observed in this study.

After treatment with study drug on POD 0, AF647-ZOL and OFS-3 treated animals were observed to have peak fluorescence signal intensity at 7 to 10 days after surgery whereas Cat K 680 FAST treated animals were observed to have peak signal intensity at approximately POD 4. The finding that these 3 different compounds show signal intensity between POD 4 and POD 10 may indicate that this timeframe corresponds with peak osteoclast activity at the site of tendon-bone repair in this model and this may have implications for future studies evaluating the timing of administration of bioactive compounds using this model. However, the observed peak signal intensity between POD 4 and POD 10 may also reflect an overlap of the pharmacokinetic properties of these compounds with the BP-containing compounds having a more similar pharmacokinetic profile. Inferences related to the timeline of osteoclast activity at the site of repair can also be made from the results using Ctsk sensitive imaging agents (i.e. Cat K 680 FAST and OFS-3). Animals in the repair group that received Cat K 680 FAST (via systemic administration) demonstrated significantly higher on-target fluorescence signal compared to the sham group at all time points except on POD 0. Similarly, on-target fluorescence signal in the repair cohorts treated with OFS-3 (local and systemic) was significantly higher than the off-target fluorescence from POD 4 to POD 10 (p<0.0015) and on POD 14 (p<0.049). However, on POD 1 in the repaired systemic OFS-3 treatment group there was no significant difference in the on-target compared to off-target signal (p=0.14).

The data supports the use of biologically-coupled bisphosphonate-targeting for the site of tendon-to-bone repair and there was minimal difference in the results whether the BP-containing compounds were administered locally or systemically. AF647-ZOL treated animals showed no significant difference in on-target signal when comparing local and systemic treatment groups at all time points studied. On-target fluorescence signal in repaired cohorts (local and systemic) treated with AF674-ZOL was also significantly higher than off-target signal in both the sham and repaired cohorts (p<0.001) until signal returned to baseline on POD 17, except for the systemic treatment group on POD 1 (p=0.31). Similarly, animals in the repair group that received Cat K 680 FAST demonstrated significantly higher on-target fluorescence signal compared to the sham group at all time points except on POD 0. Animals in repair cohorts that received either local or systemic OFS-3 demonstrated significantly higher on-target fluorescence signal compared to the sham group at all time points (p<0.001), except for the local OFS-3 treatment group at POD 1 (p=0.66).

After any limb injury, animals will typically alter normal weight bearing on their injured limb. The injured limb is subjected to an increased proportion of the animal's weight load at the forefoot relative to baseline forefoot weight bearing. This is while also decreasing the weight bearing load at the injured hindfoot presumably to avoid pain at the hindfoot incision. The other limbs are also subjected to more than their usual weight bearing load. This appears to lead to occult bone stress reactions at the forefeet of the front and hind limbs among all study animals which leads to the detection of signal after administration of the imaging probes utilized in this investigation.

Although animals in the repair groups treated with OFS-3 showed a significant decrease in on-target signal in the local treatment group compared to the systemic treatment group on POD 1, there was no significant difference after POD 1. This observation of decreased signal detected early on in the local application group could be related to administration technique where it is possible that study drug leaks out of the wound prior to skin closure potentially leading to an overall smaller initial dose than the parenteral administration route.

Animals in the Cat K 680 FAST treatment groups received 400 times the dose of the animals in the AF647-ZOL or OFS-3 treatment groups which sets a very high upper threshold for background activity from Cat K 680 FAST. Despite this large dose difference similar signals levels were achieved across these groups; however, also noteworthy was that both the peak signal and retention of signal in the Cat K 680 FAST treatment groups was shorter in duration despite the exceedingly larger doses administered.

The sham surgery animals treated with Cat K 680 FAST demonstrated significantly increased off-target signal relative to on-target signal at all time points (p<0.014). This was influenced by at least two factors: 1) as noted above, Cat K 680 Fast was administered per manufacturer recommendations at a dose 400× greater than the BP-containing compounds, thus off-target sites are likely saturated, and 2) sham animals likely exhibited decreased weight bearing at the operative limb while also lacking the physiologic stress of a mechanically disrupted calcaneus (i.e. no application of burr at the time of surgery and the subsequent infiltration of activated osteoclasts).

While the detection of increased fluorescence signal in the repair groups with each of these imaging probes at early time points is encouraging, it is also encouraging that at later timepoint (POD 14 and POD 17) there was no significant difference in on-target signal compared to off-target signal in the OFS-3 and AF647-ZOL treated repair cohorts. As discussed herein, this return to baseline fluorescence signal likely reflects either local osteoclast activity or the pharmacokinetic properties of these compounds, each of which has implications for future studies regarding the temporal administration of study drug. Targeting bioactive molecules to the site of tendon-bone repair at time points beyond the time of surgery is a distinct advantage of the BP-targeted Ctsk-coupled delivery strategy. This could allow for optimized ingress of progenitor cells to the site of repair followed by the targeted administration of growth factors signaling proliferation and/or differentiation that could greatly improve local tissue regeneration. In humans, percutaneous delivery of these specialized agents makes this translational approach highly appealing.

Off-target signal is certainly a concern in any drug discovery investigation. One explanation for the observation of signal in the uninjured limbs among animals in the study could be a physiologic response to increased load exposure at the uninjured limbs following unilateral Achilles tendon-to-bone repair. The increased weight bearing at the uninjured limbs to off-load the injured limb may result in increased bone turnover; this phenomenon would correlate well with clinical observations of athletes exposed to repetitive increased weight bearing loads leading to bone stress fracture injuries. In addition, if the etiology of this signal is increased weight bearing resulting in mild increased bone resorption, the sensitivity of these probes noted here as off-target signaling may have clinical implications as an assessment for increased bone turnover among patients or even animals (e.g. race horses) with musculoskeletal injuries as well as athletes at risk for bone stress injuries.

Although undesired off-target signal was noted in this initial study, this finding has much room for improvement and can be addressed with dose optimization studies. Off-target delivery can also be further optimized with administration technique given that there was a trend for decreased off-target signal with local administration compared to systemic administration of OFS-3, with significantly less signal noted on POD 1 (p=0.0004) and at POD 7 (p=0.0496) as well as near significance on POD 10 (p=0.058). This observed difference in off-target signal between local and systemic treatment groups may be due to improved access via direct application of BPs to the mechanically disrupted HAP minerals of the calcaneus at the time of repair. Furthermore, if using this BP targeted Ctsk-activated strategy to deliver an osteoinductive agent such as BMP-2, TGFβ, or NELL-1, then a small degree of osteogenic signal delivered to bone transiently is unlikely to be associated with significant sequelae.

Appropriate biologic cues to guide tissue regeneration is a recognized as a significant unmet need in the setting of tendon repair⁸. However, supraphysiologic dosing at the time of surgery is known to be associated with a number of well-described complications including ectopic bone, excessive inflammatory response leading to airway compromise and seroma formation which places patients at elevated risk of wound infection. In addition, as discussed herein, intra-operative delivery of bioactive molecules could be missing a critical time to trigger biological signaling after ingress and/or sufficient proliferation of local progenitor cell populations. The results obtained from the low dose administration of compounds in the study shows that a BP-targeted Ctsk-coupled delivery approach can allow for a significant decrease in administered dose where physiologically-relevant levels of bioactive molecules can be delivered at the cellular level rather than the gross tissue level.

### Conclusion

The data herein demonstrate the feasibility of using a novel bisphosphonate-based targeting and cathepsin-K-coupled delivery of molecules to the site of tendon-to-bone repair. Through guided biodistribution and temporal optimization of bioactive molecule delivery at the cellular level this approach holds great potential to improve soft tissue-to-bone healing as well as other sites of musculoskeletal injury, however, further investigation is certainly needed.

### Example 2: Manufacturing

As is the case for BP-F-Q, NELL-1 and BMP-2 was covalently bound to BP molecules via a peptide sequence that is a cleavable substrate for the cat-K enzyme using click chemistry. Click chemistry is a class of simple, atom-economy reactions commonly used for joining two molecular entities of choice. Siverino et al. J Vis Exp. 2018 Mar. 29; (133):56616.

### Example 3: In Vitro BMP Receptor Activation

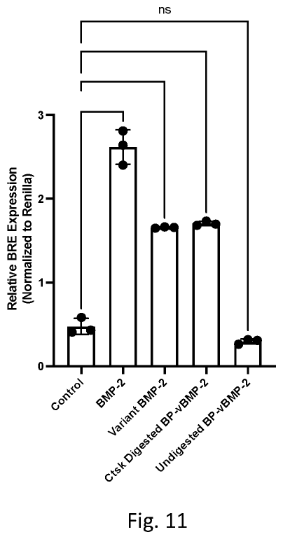

Activation of BMP receptors in vitro was assessed using a dual luciferase assay where a Rinella plasmid and BMP-responsive element (BRE) plasmid were transfected into ATDC5 cells known to express BMP receptors. These transgenic ATDC5 cells then underwent under one of the following five treatments: 1) commercially available rhBMP-2; 2) variant BMP-2; 3) Ctsk-digested BP-vBMP-2; 4) undigested BP-vBMP-2; or 5) phosphate buffered saline. Luciferase 1 expression from BRE plasmid was detected and normalized to control Rinella plasmid luciferase 2 expression (FIG. 11). These results demonstrate that Ctsk-digested BP-BMP-2 is capable of activating BMP receptors in a similar fashion to commercially available rhBMP-2 and that undigested BP-BMP-2 does not activate BMP receptors above the level of the control condition.

### Example 4: In Vitro Studies

Given that human mesenchymal stem cells (MSCs) are present at the site of human tendon repairs, human MSCs will be purchased from Lonza and maintained in culture media consisting of RPMI 1640 medium with 10% FBS for less than five passages. An equal number of cells will be plated in each culture. After 48 hours culture media will then be changed to serum-free medium containing one of the following: no additive (group 1), bisphosphonate alone (group 2), BMP-2 protein (group 3), BP-BMP-2 alone (group 4), BP-BMP-2 with cathepsin K enzyme (group 5) (see Table 4). The dose of BMP-2 will be 100 ng/ml based on previously published in vitro data (Pang S et al., Stem Cells. 2015:33(3):904-915) and this value with be used to calculate molar equivalents of BP-BMP-2 with or without cathepsin K used for groups 4 and 5. The medium will be changed every 3 days. On days 3 and 10 after transition to these experimental media solutions, proliferation assays, cell viability assays and RT-PCR assessments of gene expression for osteogenic, chondrogenic and adipogenic gene transcripts will be performed. The use of an early and late time points will provide insight regarding potential accelerated differentiation and longer-term cell viability. Cell viability will be examined using a live/dead assay kit (Thermofisher, Inc.) and cell proliferation assays will be performed using CyQUANT Assays (Thermofisher, Inc.) per manufacturer protocols. All treatment groups, assays and time points will be performed in triplicate. Total RNA will be isolated using TRIzol reagent (Invitrogen, Inc.) and stored at −80° C. Gene expression differences will be assessed via TaqMan Real-time PCR Assays (Thermofisher, Inc.) per manufacturer's protocol including genes related to osteogenic differentiation (osteopontin, osteocalcin, alkaline phosphatase), chondrogenic differentiation (Sox9, Col2A), and adipogenic differentiation (PPARγ).

TABLE 4
In vitro Study Design

| Experimental Group | Treatment |
|---|---|
| 1 | Negative Control (no added growth factor) |
| 2 | BP alone |
| 3 | BMP-2 alone |
| 4 | BP-BMP-2 |
| 5 | BP-BMP-2 + Cat K |
| 6 | NELL-1 alone |
| 7 | BP-NELL-1 |
| 8 | BP-NELL-1 + Cat K |

The same methodology will be used to assess BP, NELL-1 protein and BP-NELL-1 molecules as stated above, however, the positive control group (group 6) will use a NELL-1 dose of 800 ng/ml based on previously published in vitro data (Table 2) (Pang S et al., Stem Cells. 2015; 33(3):904-915). Again, based on this standard dose of NELL-1 calculated molar equivalents of BP-NELL-1 with or without cathepsin K will be used in groups 7 and 8 for comparison.

Statistical methods: The statistical analysis of resultant data will utilize one-way analysis of variance models (one set for the BMP-2 experiments and one set for the NELL-1 experiments). For each of set of models, the gene expression of the various genes will be the outcomes. There will be a term for treatment in these models. Transformations will be considered if the distributions of the gene expression values are non-normal and will use Tukey post-hoc tests to perform pairwise group tests.

### Example 5: In Vivo Studies

A dose of 1.25 mg/kg of recombinant human NELL-1 (rhNELL-1) administered intravenously has been demonstrated to lead to bone formation in an osteoporotic mouse model (James A W et al. Nat Commun. 2015; 6:7362). In addition, they have described the pharmacokinetics of systemically administered NELL-1-PEG, a chemically modified version of NELL-1 protein using polyethylene glycol (aka PEGylation), referred to as NELL-1-PEG, at a dose of 1.25 mg/kg administered systemically once per week. This treatment has been shown to enhance fracture healing with no abnormalities detected on toxicology testing and no evidence of ectopic bone formation radiographically or on organ retrieval analysis (Kwak J H et al. Biomaterials. 2015; 57:73-83; Tanjaya J et al. Am J Pathol. 2018; 188(3):715-727). To improve the pharmacokinetics further bone NELL-1-PEG has been covalently bound to BP molecules to make BP-NELL-1-PEG. BP-NELL-1-PEG at a dose of 10 mg/kg once per week has demonstrated an ability to preserve bone volume among mice in space (unpublished data). These results will guide the initial dosing scheme of BP-NELL-1. Systemic administration of rhBMP-2 at doses of 0.5 mg, 1 mg and 5 mg per mouse per day have been associated with statistically significant increases in total bone volume among osteopenic mice (Turgeman G et al. J Cell Biochem. 2002; 86(3):461-474). Given that mice are approximately 10% the weight of a rat, there is data to support the systemic administration of 5 mg to 10 mg of rhBMP-2 in rats. These data will guide the initial dosing of scheme of BP-BMP-2. Initial dosing pilot experiments using an Achilles tendon-bone repair model will also include post-mortem IHC staining for NELL-1 and BMP-2 proteins at the site of tendon repair versus the contralateral uninjured enthesis at 3 days after injection of BP-NELL-1 and BP-BMP-2. Pharmacokinetic assessments will be performed by labeling BP-NELL-1 and BP-BMP-2 with an amine-reactive near-infrared fluorochrome (VivoTag 680XL, Perkin Elmer) followed by daily imaging until optical signal is no longer detected using IVIS Lumina II imaging system. Dose escalation studies will be performed as needed based on initial results from pilot studies. With regard to timing, based on improved pharmacokinetics and localization associated with BP chaperones (ex. BP-NELL-1-PEG data) a single dose administered immediately following the surgical procedure will be initially investigated. Local application of these compounds, multiple systemic doses and/or delayed systemic dosing regimens will be investigated if initial single systemic dosing protocols are not efficacious.

Based on dosing data from pilot experiments the efficacy of BP-NELL-1 and BP-BMP-2 in an Achilles tendon-to-bone repair model will be characterized. Previous rat tendon repair studies have demonstrated that initial healing is completed approximately 2 weeks after surgical repair and by 6 weeks the majority of healing has occurred (Galatz L M et al. J Orthop Res. 2006; 24(3):541-550; Kremen et al. Am J Sports Med. 2019: 47(11):2737-2744; Zhao S et al. J Surg Res. 2015:193(1):33-42). Twelve groups of rats (n=15 animals/group) will undergo acute Achilles tendon-bone repair as described in Example 1. Groups TA and 1B will receive repair alone without augmentation. Groups 2A and 2B will receive systemically delivered intravenous (IV) BP alone. Groups 3A and 3B with receive rhBMP-2 protein solution delivered via interposed type I bovine collagen sponge (DuraGen, Integra LifeSciences) at the repair site. Groups 4A and 4B with receive systemic BP-BMP-2. Groups 5A and 5B with receive rhNELL-1 protein in normal saline delivered via interposed type 1 bovine collagen sponge. Groups 6A and 6B with receive systemic BP-NELL-1. Groups 1A, 2A. 3A, 4A, 5A, and 6A will be euthanized at 2 weeks post-operatively. Groups 1B, 2B, 3B. 4B, 5B, and 6B will be euthanized at 6 weeks post-operatively (see Table 5). Assessing an early and late time point will provide evidence of any accelerated healing. Following euthanasia operative limbs will be harvested. Twelve animals from each group will be dedicated to cyclic loading and load to failure testing using an Instron materials testing machine as previously described (Galatz L M et al. J Orthop Res. 2006:24(3):541-550). Three animals from each group will undergo fixation in 4% formalin, decalcified using 0.5M EDTA and subjected to hematoxylin and eosin staining, collagen birefringence microscopy and safranin O staining to evaluate for the presence of fibrocartilage.

TABLE 5
In vivo Study Design

| Experimental | | Euthenasia |
|---|---|---|
| Group | Treatment | Time Point |
| 1A | Repair alone (no augmentation) | 2 weeks |
| 1B | Repair alone (no augmentation) | 6 weeks |
| 2A | BP alone (IV administration) | 2 weeks |
| 2B | BP alone (IV administration) | 6 weeks |
| 3A | rhBMP-2 protein in collagen sponge | 2 weeks |
| 3B | rhBMP-2 protein in collagen sponge | 6 weeks |
| 4A | BP-BMP-2 (IV administration) | 2 weeks |
| 4B | BP-BMP-2 (IV administration) | 6 weeks |
| 5A | rhNELL-1 protein in collagen sponge | 2 weeks |
| 5B | rhNELL-1 protein in collagen sponge | 6 weeks |
| 6A | BP-NELL-1 (IV administration) | 2 weeks |
| 6B | BP-NELL-1 (IV administration) | 6 weeks |

Statistical methods: The statistical analysis of resultant data will utilize two-way analysis of variance models. The outcomes assessed in these models will be cyclic loading and load to failure. For each model the terms in the model will include treatment (see Table 5), sacrifice time (2 weeks, 6 weeks) and the treatment by time interaction effect. A term for the main effect of sex in these models will be added as well as interaction effects with sex (ex. sex by treatment interaction). Transformations will be considered if the distributions of the biomechanics values are non-normal and Tukey post-hoc tests will be used to perform pairwise group tests. With the sample sizes of 12 per group (6 male rats and 6 female rats) in each cell of 2×6 factorial experiment, there will be 80% power to detect effect sizes of at least 1.2 for the time effect within treatments and 0.83 between pairs of treatments assuming an ANOVA analysis and two-sided 0.05 level of significance.

### Example 6: PTH and PTHrP

PTH and PTHrP are both proteins that could be synthesized using a similar technique to the approach to modified BMP-2 synthesis (as described by Silverino C, et al JOVE 2018, PMID: 29658921). This would allow for incorporation of an unnatural amino acid that is amenable to Click chemistry which allows for conjugation of these bioactive moieties to our BP-cathepsin K-coupled molecular cargo delivery approach. Following this the effects of BP-(Ctsk-sensitive peptide)-PTH or BP-(Ctsk-sensitive peptide)-PTHrP compounds will be assessed in vivo using animal models of fracture healing, effect on subchondral bone and cartilage tissues using an osteoarthritis (OA) model, soft tissue-to-bone healing, and osteointegration of implants using mechanical testing as described in Morinaga, et al., Biomaterials vol. 192 (2019): 62-74.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Rothrauff B B, Tuan R S. 2014. Cellular therapy in bone-tendon interface regeneration. Organogenesis 10:13-28.
2. Kuhn J E, Dunn W R, Sanders R, et al. 2013. Effectiveness of physical therapy in treating atraumatic full-thickness rotator cuff tears: a multicenter prospective cohort study. J Shoulder Elbow Surg 22:1371-1379.
3. Mather R C, 3rd, Koenig L, Acevedo D. et al. 2013. The societal and economic value of rotator cuff repair. J Bone Joint Surg Am 95:1993-2000.
4. Galatz L M, Ball C M, Teefey S A, et al. 2004. The outcome and repair integrity of completely arthroscopically repaired large and massive rotator cuff tears. J Bone Joint Surg Am 86-A:219-224.
5. Slabaugh M A, Nho S J, Grumet R C, et al. 2010. Does the literature confirm superior clinical results in radiographically healed rotator cuffs after rotator cuff repair? Arthroscopy 26:393-403.
6. Nichols A E C, Best K T, Loiselle A E. 2019. The cellular basis of fibrotic tendon healing: challenges and opportunities. Transl Res 209:156-168.
7. Smith L, Xia Y, Galatz L M, et al. 2012. Tissue-engineering strategies for the tendon/ligament-to-bone insertion. Connect Tissue Res 53:95-105.
8. Kovacevic D. Rodeo S A. 2008. Biological augmentation of rotator cuff tendon repair. Clin Orthop Relat Res 466:622-633.
9. Lee J S, Kim T W, Park S, et al. 2015. Reduction of Adipose Tissue Formation by the Controlled Release of BMP-2 Using a Hydroxyapatite-Coated Collagen Carrier System for Sinus-Augmentation/Extraction-Socket Grafting. Materials (Basel) 8:7634-7649.
10. Bez M, Kremen T J, Tawackoli W, et al. 2018. Ultrasound-Mediated Gene Delivery Enhances Tendon Allograft Integration in Mini-Pig Ligament Reconstruction. Mol Ther 26:1746-1755.
11. Hashimoto Y, Yoshida G, Toyoda H, et al. 2007. Generation of tendon-to-bone interface “enthesis” with use of recombinant BMP-2 in a rabbit model. J Orthop Res 25:1415-1424.
12. Martinek V, Latterman C, Usas A, et al. 2002. Enhancement of tendon-bone integration of anterior cruciate ligament grafts with bone morphogenetic protein-2 gene transfer: a histological and biomechanical study. J Bone Joint Surg Am 84-A:1123-1131.
13. Rundle C H, Chen S T, Coen M J, et al. 2014. Direct lentiviral-cyclooxygenase 2 application to the tendon-bone interface promotes osteointegration and enhances return of the pull-out tensile strength of the tendon graft in a rat model of biceps tenodesis. PLoS One 9:e98004.
14. Kim H M, Galatz L M, Das R, et al. 2011. The role of transforming growth factor beta isoforms in tendon-to-bone healing. Connect Tissue Res 52:87-98.
15. Wang R, Xu B. Xu H G. 2017. Up-Regulation of TGF-3 Promotes Tendon-to-Bone Healing after Anterior Cruciate Ligament Reconstruction using Bone Marrow-Derived Mesenchymal Stem Cells through the TGF-β/MAPK Signaling Pathway in a New Zealand White Rabbit Model. Cell Physiol Biochem 41:213-226.
16. Lu H H, Thomopoulos S. 2013. Functional attachment of soft tissues to bone: development, healing, and tissue engineering. Annu Rev Biomed Eng 15:201-226.
17. Killian M L, Cavinatto L, Shah S A, et al. 2014. The effects of chronic unloading and gap formation on tendon-to-bone healing in a rat model of massive rotator cuff tears. J Orthop Res 32:439-447.
18. Galatz L M. Rothermich S Y, Zaegel M, et al. 2005. Delayed repair of tendon to bone injuries leads to decreased biomechanical properties and bone loss. J Orthop Res 23:1441-1447.
19. Sato E J, Killian M L, Choi A J, et al. 2015. Architectural and biochemical adaptations in skeletal muscle and bone following rotator cuff injury in a rat model. J Bone Joint Surg Am 97:565-573.
20. Richard E T, Morinaga K, Zheng Y, et al. 2021. Design and Synthesis of Cathepsin-K-Activated Osteoadsorptive Fluorogenic Sentinel (OFS) Probes for Detecting Early Osteoclastic Bone Resorption in a Multiple Myeloma Mouse Model. Bioconjug Chem 32:916-927.
21. Rosenberg L. 1971. Chemical basis for the histological use of safranin O in the study of articular cartilage. J Bone Joint Surg Am 53:69-82.
22. Littlewood-Evans A. Kokubo T, Ishibashi 0, et al. 1997. Localization of cathepsin K in human osteoclasts by in situ hybridization and immunohistochemistry. Bone 20:81-86.
23. Benglis D, Wang M Y, Levi A D. 2008. A comprehensive review of the safety profile of bone morphogenetic protein in spine surgery. Neurosurgery 62: ONS423-431; discussion ONS431.
24. Xu J, Su W, Chen J, et al. 2021. The Effect of Antiosteoporosis Therapy With Risedronate on Rotator Cuff Healing in an Osteoporotic Rat Model. Am J Sports Med 49:2074-2084.
25. Lui P P, Lee Y W, Mok T Y, et al. 2013. Alendronate reduced peri-tunnel bone loss and enhanced tendon graft to bone tunnel healing in anterior cruciate ligament reconstruction. Eur Cell Mater 25:78-96.
26. Hjorthaug G A, Soreide E, Nordsletten L, et al. 2018. Negative effect of zoledronic acid on tendon-to-bone healing. Acta Orthop 89:360-366.
27. Thomopoulos S, Matsuzaki H, Zaegel M. et al. 2007. Alendronate prevents bone loss and improves tendon-to-bone repair strength in a canine model. J Orthop Res 25:473-479.
28. Sung C M, Kim R J, Hah Y S, et al. 2020. In vitro effects of alendronate on fibroblasts of the human rotator cuff tendon. BMC Musculoskelet Disord 21:19.
29. Spoendlin J, Meier C, Jick S S, et al. 2016. Bisphosphonate therapy start may transiently increase the risk of tendon rupture in patients with glucocorticoid co-medication: a population-based observational study. Pharmacoepidemiol Drug Saf 25:1116-1123.
30. Gordon D H. 2005. Efficacy and safety of intravenous bisphosphonates for patients with breast cancer metastatic to bone: a review of randomized, double-blind, phase III trials. Clin Breast Cancer 6:125-131.
31. Papagiannopoulou D. 2017. Technetium-99m radiochemistry for pharmaceutical applications. J Labelled Comp Radiopharm 60:502-520.
32. Hokugo A, Kanayama K, Sun S, et al. 2019. Rescue bisphosphonate treatment of alveolar bone improves extraction socket healing and reduces osteonecrosis in zoledronate-treated mice. Bone 123:115-128.
33. Okawa H, Kondo T, Hokugo A, et al. 2022. Mechanism of bisphosphonate-related osteonecrosis of the jaw (BRONJ) revealed by targeted removal of legacy bisphosphonate from jawbone using competing inert hydroxymethylene diphosphonate. Elife 11.

We claim:

1. A molecule comprising:
i) a bioactive moiety;
ii) a cleavable linker; and
iii) a bisphosphonate (BP) moiety;
wherein the BP moiety has a P-C-P structure, and
wherein the cleavable linker links the bioactive moiety to the BP moiety.

2. The molecule of claim 1, wherein the bioactive moiety is an osteochondral factor, an osteogenic factor, a tenogenic factor or an Extracellular Matrix (ECM) protein.

3. The molecule of claim 2, wherein the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP.

4. The molecule of claim 3, wherein the osteochondral factor is NELL-1 or BMP-2.

5. The molecule of claim 2, wherein the tenogenic factor is TGFβ-1, TGFβ-2, TGF-β3, FGF, CTGF, BMP-12, BMP-13, BMP-14, CCN1 or WISP-1.

6. The molecule of claim 5, wherein the tenogenic factor is TGFβ-1 or TGFβ-2.

7. The molecule of claim 2, wherein the ECM protein is a collagen, elastin, fibrin, fibronectin, gelatin, laminin, integrin, CCN1, WISP-1 or CTGF.

8. The molecule of claim 1, wherein the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

9. The molecule of any one of claims 1 to 8, wherein the cleavable linker is a cathepsin-K-sensitive peptide linker.

10. The molecule of claim 9, wherein the cathepsin-K-sensitive peptide linker is GHPGGPQG (SEQ ID NO: 1) or GGGMGPSGPWGGK (SEQ ID NO: 2).

11. The molecule of any one of claims 1 to 8, wherein the cleavable linker is an MMP cleavable linker.

12. The molecule of claim 11, wherein the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

13. The molecule of any one of claims 1 to 8, wherein the cleavable linker is a pH sensitive linker.

14. The molecule of any one of claims 1 to 13, wherein the BP moiety does not inhibit osteoclast function and/or only minimally inhibits farnesyl pyrophosphate synthestase (FPSS).

15. The molecule of any one of claims 1 to 14, wherein the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

16. A composition comprising the molecule of any one of claims 1 to 15.

17. A pharmaceutical composition comprising the molecule of any one of claims 1 to 15 or the composition of claim 16.

18. A method of targeting a molecule to a site of injury to a subject in need thereof, comprising administering a molecule comprising:
i) a bioactive moiety;
ii) a cleavable linker; and
iii) a bisphosphonate (BP) moiety;
wherein the BP moiety has a P-C-P structure, and
wherein the cleavable linker links the bioactive moiety to the BP moiety.

19. The method of claim 18, wherein the site of injury is enthesis tissue.

20. The method of claim 18 or claim 19, wherein the site of injury is at a rotator cuff, a distal biceps tendon, a pectoralis major, a patellar tendon, a quadriceps tendon, or a triceps tendon.

21. The method of claim 18 or claim 19, wherein the site of injury is also a site of surgery and the surgery is an anterior cruciate ligament (ACL) reconstruction, a posterior cruciate ligament (PCL) reconstruction, a lateral collateral ligament (LCL) or medial collateral ligament (MCL) repair/reconstruction, a medial patellofemoral ligament reconstruction, a meniscus root repair, a meniscus allograft transplantation, or an osteochondral allograft transplantation.

22. The method of any one of claims 18 to 21, wherein the site of injury is at the site of osteointegration of implantable prosthesis or osteointegration of allograft tissue.

23. The method of any one of claims 18 to 22, wherein the molecule is administered systemically.

24. The method of any one of claims 18 to 22, wherein the molecule is administered locally.

25. The method of any one of claims 18 to 24, wherein the bioactive moiety is an an osteochondral factor, an osteogenic factor, a tenogenic factor or an Extracellular Matrix (ECM) protein.

26. The method of claim 25, wherein the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP.

27. The method of claim 26, wherein the osteochondral factor is NELL-1 or BMP-2.

28. The method of claim 25, wherein the tenogenic factor is TGFβ-1, TGFβ-2, TGF-β3, FGF, CTGF, BMP-12, BMP-13, BMP-14, CCN1 or WISP-1.

29. The method of claim 28, wherein the tenogenic factor is TGFβ-1 or TGFβ-2.

30. The method of claim 25, wherein the ECM protein is a collagen, elastin, fibrin, fibronectin, gelatin, laminin, integrin, CCN1, WISP-1 or CTGF.

31. The method of any one of claims 18 to 24, wherein the bioactive moiety is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

32. The method of any one of claims 18 to 31, wherein the cleavable linker is a cathepsin-K-sensitive peptide linker.

33. The method of claim 32, wherein the cathepsin-K-sensitive peptide linker is GHPGGPQG (SEQ ID NO: 1) or GGGMGPSGPWGGK (SEQ ID NO: 2).

34. The method of any one of claims 18 to 31, wherein the cleavable linker is a MMP cleavable linker.

35. The method of claim 34, wherein the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

36. The method of any one of claims 18 to 31, wherein the cleavable linker is a pH sensitive linker.

37. The method of any one of claims 18 to 36, wherein the BP moiety does not inhibit osteoclast function and/or only minimally inhibits farnesyl pyrophosphate synthestase (FPSS).

38. The method of any one of claims 18 to 37, wherein the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).

39. A method of treating a tendon injury, fracture and/or bone stress injury and/or promoting soft tissue to bone healing to a subject in need thereof, comprising administering a molecule comprising:
i) a bioactive moiety;
ii) a cleavable linker; and
iii) a bisphosphonate (BP) moiety;
wherein the BP moiety has a PCP structure, and
wherein the cleavable linker links the bioactive moiety to the BP moiety.

40. The method of claim 39, wherein the tendon injury is a rotator cuff, a distal biceps tendon, pectoralis major, patellar tendon, quadriceps tendon, and/or triceps tendon.

41. A method of enhancing incorporation of allograft tissues or promoting repair of enthesis during or after an orthopedic surgical procedure to a subject in need thereof comprising administering a molecule comprising:
i) a bioactive moiety;
ii) a cleavable linker; and
iii) a bisphosphonate (BP) moiety;
wherein the BP moiety has a PCP structure, and
wherein the cleavable linker links the bioactive moiety to the BP moiety.

42. The method of claim 41, wherein the orthopedic surgical procedure is an osteotomy, an ACL reconstruction, a PCL reconstruction, an LCL or MCL repair/reconstruction, a medial patellofemoral ligament reconstruction, a meniscus root repair, a meniscus allograft transplantation, and/or osteochondral allograft transplantation.

43. The method of claim 41 or claim 42, wherein the molecule is administered just before the orthopedic surgical procedure.

44. The method of claim 41 or claim 42, wherein the molecule is administered during the orthopedic surgical procedure.

45. The method of claim 41 or claim 42, wherein the molecule is administered after the orthopedic surgical procedure.

46. A method of treating osteoporosis, osteoarthritis and/or cartilage defects in a subject in need thereof, comprising administering a molecule comprising:
i) a bioactive moiety;
ii) a cleavable linker; and
iii) a bisphosphonate (BP) moiety;
wherein the BP moiety has a PCP structure, and
wherein the cleavable linker links the bioactive moiety to the BP moiety.

47. The method of any one of claims 39 to 46, wherein the molecule is administered systemically.

48. The method of any one of claims 39 to 46, wherein the molecule is administered locally at the site of the tendon injury or surgical procedure.

49. The method of any one of claims 39 to 48, wherein the bioactive moiety is an osteochondral factor, an osteogenic factor, a tenogenic factor or an Extracellular Matrix (ECM) protein.

50. The method of claim 49, wherein the osteochondral factor is NELL-1, TGF-β1, TGF-β2, TGF-β3, BMP-1, BMP-2, BMP-3, BMP-4, BMP-5, BMP-6, BMP-7, BMP-8a, BMP-8b, BMP-10, BMP-11, BMP-15, or rhBMP.

51. The method of claim 50, wherein the osteochondral factor is NELL-1 or BMP-2.

52. The method of claim 49, wherein the tenogenic factor is TGFβ-1, TGFβ-2, TGF-β3, FGF, CTGF, BMP-12, BMP-13, BMP-14, CCN1 or WISP-1.

53. The method of claim 52, wherein the tenogenic factor is TGFβ-1 or TGFβ-2.

54. The method of claim 49, wherein the ECM protein is a collagen, elastin, fibrin, fibronectin, gelatin, laminin, integrin, CCN1, WISP-1 or CTGF.

55. The method of any one of claims 39 to 48, wherein the bioactive agent is parathyroid hormone, parathyroid hormone-related protein, growth differentiation factor 11 (GDF11), activin receptor-like kinase-1-Fc (ALK1-Fc), or activin receptor-like kinase-4-Fc (ALK4-Fc).

56. The method of any one of claims 39 to 55, wherein the cleavable linker is a cathepsin-K-sensitive peptide linker.

57. The method of claim 56, wherein the cathepsin-K-sensitive peptide linker is GHPGGPQG (SEQ ID NO: 1) or GGGMGPSGPWGGK (SEQ ID NO: 2).

58. The method of any one of claims 39 to 55, wherein the cleavable linker is a MMP cleavable linker.

59. The method of claim 58, wherein the MMP cleavable linker is PLGLAG (SEQ ID NO: 3) or Ac-GCRD-GPQGIWGQ-DRCG (SEQ ID NO: 4) or Ac-GCRD-GPQGIAGQ-DRCG (SEQ ID NO: 5).

60. The method of any one of claims 39 to 55, wherein the cleavable linker is a pH sensitive linker.

61. The method of any one of claims 39 to 60, wherein the BP moiety does not inhibit osteoclast function and/or only minimally inhibits farnesyl pyrophosphate synthestase (FPSS).

62. The method of any one of claims 39 to 61, wherein the BP moiety is 2-(pyridin-4-yl)ethane-1,1-diylbisphosphonic acid (p-PyrEBP), 1-hydroxy-2-(pyridin-4-yl)ethane-1,1-diylbisphosphonic acid (p-RIS), methylene bisphosphonate (MBP), methylene hydroxyl bisphosphonate (MHDP), etidronate (EHDP), methylhydroxyl diphosphonate, clodronate, isclodronate, tiludronate, 2-hydroxy-2-phosphono-3-(pyridine-3-yl)propanoic acid (3-PEHPC), or 2-hydroxy-3-(imidazo[1,2-c]pyridine-3-yl)-2-phosphonopropanoic acid (3-IP-EHPC).
